(19) United States
(12) Patent Application Publication
(10) Pub. No.: US 2026/0344985 A1
(43) Pub. Date: Nov. 5, 2026

(54) PARTIALLY REUSABLE MEDICAL DEVICES AND RELATED METHODS OF USE

(71) Applicants: Boston Scientific Scimed, Inc., Maple Grove, MN (US); Boston Scientific Medical Device Limited, Galway (IE)

(72) Inventors: Sean Christopher POWELL, Holden, MA (US); Joseph Phillip ALBINA, Hudson, MA (US); Shrikant Vasant RAUT, Pune (IN); Pooja Bhuvanesh KULKARNI, Pune (IN); Aditya DHANOTIYA, Pune (IN)

(73) Assignees: Boston Scientific Scimed, Inc., Maple Grove, MN (US); Boston Scientific Medical Device Limited, Galway (IE)

(21) Appl. No.: 19/662,680

(22) Filed: Apr. 29, 2026

Related U.S. Application Data
(60) Provisional application No. 63/798,761, filed on May 2, 2025.
(60) Provisional application No. 63/862,208, filed on Aug. 12, 2025.

(51) Int. Cl. A61B 1/005; A61B 1/00; A61B 1/018

(52) U.S. Cl. CPC A61B 1/0052; A61B 1/00042; A61B 1/00114; A61B 1/00119; A61B 1/00128; A61B 1/0057; A61B 1/018

(57) ABSTRACT

A medical device may include a first portion and a second portion removably coupled to the first portion. The first portion may include a housing, an actuator, and a gear assembly coupled to the actuator. The second portion may include a housing connected to the housing of the first portion, a shaft extending distally from the housing of the second portion and defining a working channel, and a spool assembly coupled to a first pull wire and a second pull wire. Each of the first pull wire and the second pull wire may extend through the shaft to a distal end of the shaft. The actuator may be coupled to the spool assembly via the gear assembly to deflect the distal end of the shaft.

### CROSS-REFERENCE TO RELATED APPLICATIONS

This application claims the benefit of priority to U.S. Provisional Application No. 63/798,761, filed on May 2, 2025, and U.S. Provisional Application No. 63/862,208, filed on Aug. 12, 2025, each of which is incorporated by reference herein in its entirety.

### TECHNICAL FIELD

Various aspects of this disclosure relate generally to partially reusable medical devices and related methods of use. In particular, aspects of this disclosure relate to partially reusable medical devices including a reusable portion removably coupled to a disposable portion having a deflectable shaft.

### BACKGROUND

A scope device, such as a duodenoscope, an endoscope, or other scope device, may include a shaft coupled to a handle including actuators or controls. The shaft may include a working channel and a deflectable distal end. During a medical procedure, the shaft may be inserted into a body lumen of a subject (e.g., a patient). The actuators or controls on the handle may be used to deflect the distal end of the shaft to, e.g., change an orientation of a medical instrument extending through the working channel or a view provided by an imaging device at the distal end of the shaft. After the medical procedure, a reusable scope device may be reused after being disinfected. A disposable scope device may be disposed of in its entirety after the medical procedure. However, disposable and reusable scope devices may present challenges. For example, disposable scope devices may have high costs or increase medical waste. Reusable scope devices may be difficult to disinfect, which may increase a patient risk of infection.

### SUMMARY

Each of the aspects disclosed herein may include one or more aspects of the features described in connection with any of the other disclosed aspects.

In an aspect, a medical device may include a first portion and a second portion removably coupled to the first portion. The first portion may include a housing, an actuator, and a gear assembly coupled to the actuator. The second portion may include a housing connected to the housing of the first portion, a shaft extending distally from the housing of the second portion and defining a working channel, and a spool assembly coupled to a first pull wire and a second pull wire. Each of the first pull wire and the second pull wire may extend through the shaft to a distal end of the shaft. The actuator may be coupled to the spool assembly via the gear assembly to deflect the distal end of the shaft.

In some aspects, each of the first portion and the second portion may include an umbilicus. The umbilicus of the first portion may be configured to transmit electrical signals, receive electrical signals, or deliver power to the medical device. The umbilicus of the second portion may be configured to couple to a source of fluid or a source of suction. The spool assembly may include a spool and a gear. The gear of the spool assembly may be fixedly coupled to the spool.

In some aspects, the gear assembly may include a first gear, a second gear, and a third gear. The first gear may be fixedly coupled to the actuator, the second gear may be disposed between and engaged with the first gear and the third gear, and the third gear may be disposed between and engaged with the second gear and the gear of the spool assembly. In some aspects, the first gear may be aligned with the second gear and the third gear. In other aspects, the first gear may be offset from the second gear and the third gear.

In some aspects, the gear assembly may include a first gear and a gear unit comprising a plurality of gears. The first gear may be fixedly coupled to the actuator. The gear unit may be disposed between the first gear and the gear of the spool assembly. The gear unit may include a first compound gear, a second compound gear, and a single gear disposed between the first compound gear and the second compound gear. The gear unit may be laterally movable relative to the first gear to adjust a gear ratio of the gear assembly.

In other aspects, the gear assembly may include a first gear and a rack gear. The first gear may be fixedly coupled to the actuator. A proximal end of the rack gear may be engaged with the first gear, and a distal end of the rack gear may be engaged with the gear of the spool assembly.

In some aspects, the first portion may include a first shaft within the housing of the first portion. The gear assembly may include a first gear and a second gear positioned perpendicularly to one another. The first gear may be fixedly coupled to the actuator, and the second gear may be fixedly coupled to the first shaft. The second portion may include a second shaft within the housing of the second portion and that may be coupled to the spool assembly. The second shaft may be rotatable with the first shaft to rotate the spool assembly. In some aspects, a cross-sectional shape of the first shaft may be non-round, and the second shaft may define a lumen having a cross-sectional shape complementary to the cross-sectional shape of the second shaft. The first shaft may be received within the lumen of the second shaft. In other aspects, the first portion may include a first coupler fixedly coupled to a distal end of the first shaft, and the second portion may include a second coupler fixedly coupled to a proximal end of the second shaft. The first coupler may be engaged with the second coupler. In other aspects, the first portion may include a second shaft and a belt assembly within the housing of the first portion. The second portion may include a third shaft coupled to the spool assembly within the housing of the second portion. The first shaft may be coupled to the second shaft via the belt assembly. The third shaft may be rotatable with the second shaft to rotate the spool assembly.

In an aspect, a medical device may include a first portion and a second portion removably coupled to the first portion. The first portion may include an actuator and

a first gear rotatably fixedly coupled to the actuator. The second portion may include a shaft, a spool assembly, a first pull wire, and a second pull wire. The spool assembly may include a second gear fixedly coupled to a spool. Each of the first pull wire and the second pull wire may extend distally from the spool to a distal end of the shaft. The actuator may be coupled to the spool assembly to deflect the distal end of the shaft.

In some aspects, the first portion may include a third gear and a fourth gear. The third gear may be disposed between the first gear and the fourth gear, and the fourth gear may be disposed between the third gear and the second gear. In some aspects, the first portion may include a rack gear, and the rack gear may be engaged with the first gear and the second gear. In other aspects, the first portion may include a third gear and a first shaft extending parallel to a longitudinal axis of the first portion. The third gear may be positioned in contact with and perpendicular to the first gear. The second portion may include a second shaft coupled to the first shaft and the spool assembly.

In an aspect, a medical device may include a first portion and a second portion removably coupled to the first portion. The first portion may include a housing, an actuator, a first gear within the housing of the first portion, and a second gear that engages with the first gear. The first gear may be rotatably fixedly coupled to the actuator. The second portion may include a third gear within the housing of the second portion, and a pull wire. The pull wire may be coupled to the first gear. The first gear may be rotatable via the actuator to indirectly rotate the third gear to actuate the pull wire.

### BRIEF DESCRIPTION OF FIGURES

The accompanying drawings, which are incorporated in and constitute a part of this application, illustrate exemplary aspects of this disclosure and, together with the description, serve to explain the principles of this disclosure.

FIGS. 1A and 1B illustrate an exemplary medical device, in accordance with some aspects of this disclosure.

FIG. 2A illustrates an exemplary medical device including a reusable portion and a disposable portion, in accordance with some aspects of this disclosure.

FIGS. 2B-2G illustrate features of the disposable portion of the medical device of FIG. 2A, in accordance with some aspects of this disclosure.

FIGS. 2H-2M illustrate features of the reusable portion of the medical device of FIG. 2A, in accordance with some aspects of this disclosure.

FIG. 2N illustrates features of a handle of the medical device of FIG. 2A, in accordance with some aspects of this disclosure.

FIGS. 3A-3B illustrate features of an exemplary medical device handle including a reusable portion and the disposable portion of the medical device of FIG. 2A, in accordance with some aspects of this disclosure.

FIGS. 4A-4B illustrate features of another exemplary medical device handle including a reusable portion and the disposable portion of the medical device of FIG. 2A, in accordance with some aspects of this disclosure.

FIGS. 5A-5B illustrate features of a further exemplary medical device handle including a reusable portion and the disposable portion of the medical device of FIG. 2A, in accordance with some aspects of this disclosure.

FIG. 6 illustrates features of another exemplary medical device including a reusable portion and a disposable portion, in accordance with some aspects of this disclosure.

FIGS. 7A-7G illustrate features of exemplary grip portions for the disposable portion of the medical devices of FIGS. 2A, 3A-3B, 4A-4B, 5A-5B, and 6, in accordance with some aspects of this disclosure.

FIGS. 8A-8D illustrate features of an exemplary gear assembly for the reusable portion of the medical devices of FIGS. 2A, 3A, 4A, and 5A, in accordance with some aspects of this disclosure.

FIG. 9 illustrates features of an exemplary medical device including a reusable portion and a disposable portion, in accordance with some aspects of this disclosure.

FIG. 10 illustrates a schematic view of features of another exemplary medical device including a reusable portion and a disposable portion, in accordance with some aspects of this disclosure.

FIG. 11 illustrates a schematic view of features of a further exemplary medical device including a reusable portion and a disposable portion, in accordance with some aspects of this disclosure.

FIG. 12 illustrates a schematic view of features of an exemplary medical device including a reusable portion and a disposable portion, in accordance with some aspects of this disclosure.

FIG. 13 illustrates a schematic view of features of another exemplary medical device including a reusable portion and a disposable portion, in accordance with some aspects of this disclosure.

FIGS. 14A and 14B illustrate schematic views of features of a further exemplary medical device including a reusable portion and a disposable portion in a first state (FIG. 14A) and in a second state (FIG. 14B), in accordance with some aspects of this disclosure.

FIG. 15 illustrates a schematic view of features of another exemplary medical device including a reusable portion and a disposable portion, in accordance with some aspects of this disclosure.

FIG. 16A illustrates a schematic view of features of yet another exemplary medical device including a reusable portion and a disposable portion, in accordance with some aspects of this disclosure.

FIGS. 16B and 16C illustrate features of an exemplary disposable portion, in accordance with some aspects of this disclosure.

FIGS. 17A-17E illustrate features of an exemplary medical device handle, including a grip, in accordance with some aspects of this disclosure.

### DETAILED DESCRIPTION

Particular aspects of the disclosure are described in greater detail below. The terms and definitions provided herein control, if in conflict with terms or definitions incorporated by reference. Wherever possible, the same or similar reference numbers will be used through the drawings to refer to the same or like parts.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “or” is used disjunctively, such that “at least one of A or B” includes, (A), (B), (A and A), (A and B), etc. The term “or” also includes “and/or.” Relative terms such as “about,” “substantially,” and “approximately,” etc., are used to indicate a possible variation of ±10% of the stated numeric value or range.

This disclosure includes medical devices having a reusable portion (e.g., a first portion or a proximal portion) removably coupled to a disposable portion (e.g., a second portion or a distal portion). According to some aspects of this disclosure, features of the reusable portion and the disposable portion may allow the reusable portion to be, e.g., easily connected to or disconnected from the disposable portion (e.g., physically or operably). Although aspects of embodiments are described separately, it will be appreciated that they may be combined in any suitable manner.

According to some aspects of this disclosure, the disposable portion of the medical device may include components that may be difficult to disinfect or clean. For example, the disposable portion of the medical device may include a deflectable shaft, one or more channels, one or more ports in communication with the channel(s), steering or other types of control wires, pulleys or cams, or fluidic components (e.g., valves, tubing, or conduits for air, water, or suction).

According to some aspects of this disclosure, the reusable portion may include control knobs coupled to a gear mechanism within the reusable portion. The gear mechanism within the reusable portion may be configured to interact or engage with a pull wire mechanism (e.g., steering pulleys or cams) within the disposable portion to deflect a distal end of the shaft. Features of the reusable portion may improve ergonomics or usability of the medical device. For example, features of the control knobs and the gear mechanism may be customizable according to a user’s (e.g., operator’s) preference. For example, the control knobs described herein may vary in size (e.g., vary in diameter), shape, or material. For example, the medical devices herein may include various grip or grip portions that may provide a user with different handle or grip sizes.

Additionally, the gear mechanisms described herein may have varying gear ratios to, e.g., provide different operating torques so that an operator may exert more or less force on knobs to achieve a same amount of steering, subject to operator preferences. This disclosure also describes various other mechanisms that may be coupled to the control knobs of the reusable portion for deflecting the distal end of the shaft of the disposable portion.

Although duodenoscopes are illustrated herein, it will be appreciated that the disposable portion described herein may be adapted to other scope devices, such as bronchoscopes, ureteroscopes, endoscopes, gastroscopes, endoscopic ultrasonography (“EUS”) scopes, colonoscopes, laparoscopes, cystoscopes, aspiration scopes, or any other type of scope device. It will be appreciated that although the reusable and disposable portions described herein may provide a four-way steering medical device, the reusable portion or the disposable portion may be adapted to provide a two-way steering medical device. In some aspects, the same reusable portion may be used with a variety of different types of disposable portions (e.g., the same reusable portion may be used with a side-viewing duodenoscope and a forward-facing gastroscope or any other type of scope).

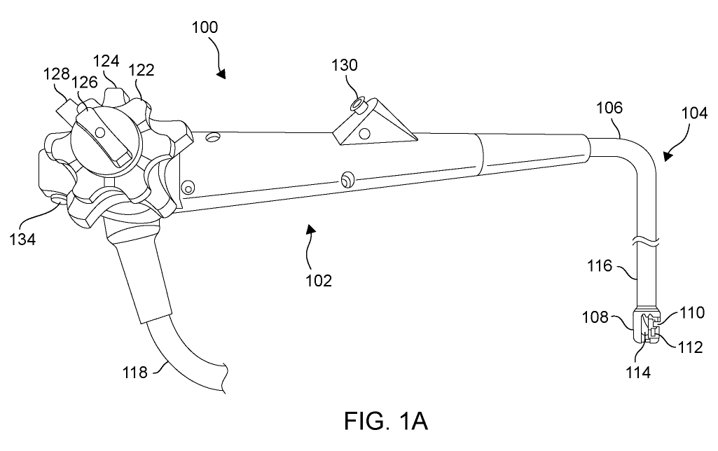

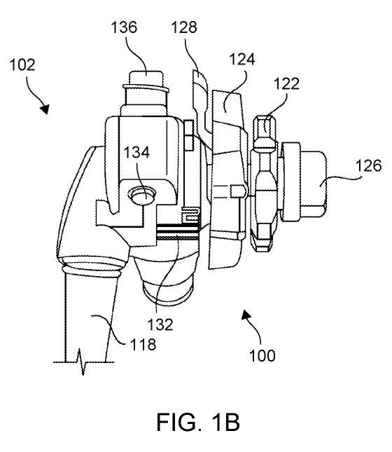

An exemplary medical device 100 (e.g., a duodenoscope) will now be described with reference to FIGS. 1A and 1B. Medical device 100 may include a handle 102 and an insertion portion 104. Insertion portion 104 may include a shaft 106 and a distal tip 108. Shaft 106 may include a steerable section 116 that is connected to distal tip 108 for deflecting distal tip 108 in up, down, left, or right directions. Steerable section 116 may be, e.g., an articulation joint. Medical device 100 may include a working channel extending through shaft 106, and a distal opening at distal tip 108 in communication with the working channel.

Distal tip 108 may include an imaging device 110 (e.g., a camera) and a light source 112 (e.g., an LED or an optical fiber). Distal tip 108 may be side facing (i.e., side viewing). That is, imaging device 110 and light source 112 may face radially outward, perpendicularly, approximately perpendicularly, or otherwise transverse to a longitudinal axis of shaft 106 and distal tip 108. Alternatively, distal tip 108 may be forward-facing (e.g., imaging device 110 or light source 112 may face distally).

Distal tip 108 may include an elevator 114 for changing an orientation of a medical tool inserted into the working channel of medical device 100. Elevator 114 may alternatively be referred to as a swing stand, pivot stand, raising base, or any other suitable term. Elevator 114 may be rotatable via, e.g., an actuation wire or another control element that extends from handle 102, through shaft 106, to elevator 114.

Handle 102 may include a first control knob 122 and a second control knob 124 for controlling steerable section 116. For example, first control knob 122 may provide left/right control of steerable section 116, and second control knob 124 may provide up/down control of steerable section 116. Handle 102 may include a first locking mechanism 126 and a second locking mechanism 128 (e.g., knobs or levers) for locking first and second control knobs 122, 124 in a desired position, and thus preventing steering of steerable section 116 in at least one of an up, down, left, or right direction. Handle 102 may include a control lever 132 (shown in FIG. 1B) for raising or lowering elevator 114.

Handle 102 may include a port 130 in communication with the working channel of medical device 100. An operator may insert a medical tool into port 130 and may extend the medical tool distally through the working channel of medical device 100. Handle 102 may include one or more control valves 136 for suction or fluid (e.g., air or water) supply. Handle 102 may include an image capture button 134 that may allow an operator to capture video or still images from imaging device 110.

Medical device 100 may include an umbilicus 118 for purposes of connecting medical device 100 to one or more auxiliary devices. The one or more auxiliary devices may include a controller or control system, an imaging system, a power supply, a fluid supply, a suction/vacuum source, or a display.

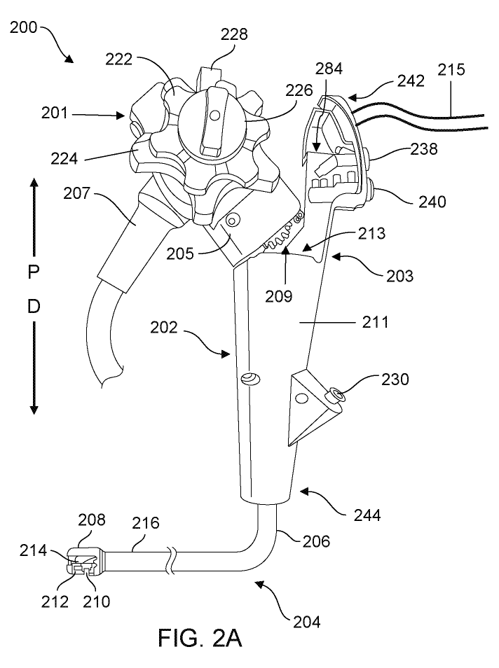

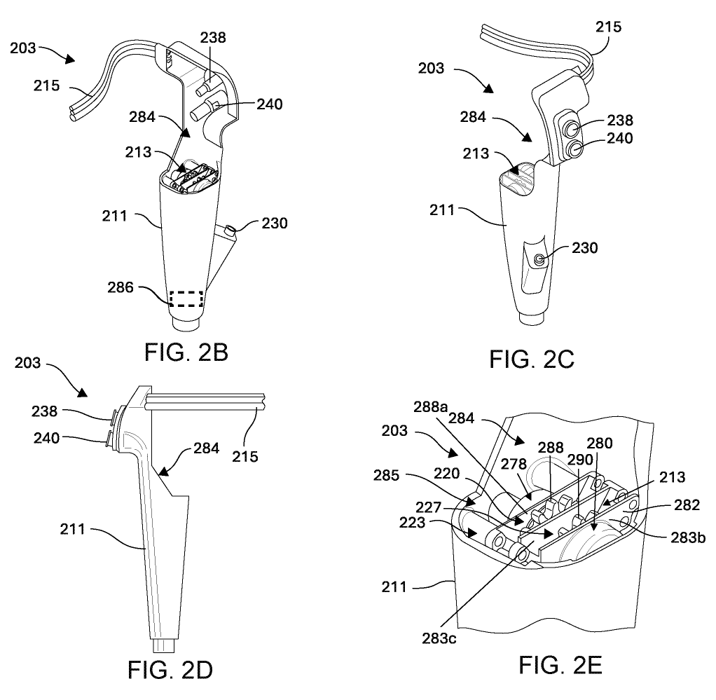

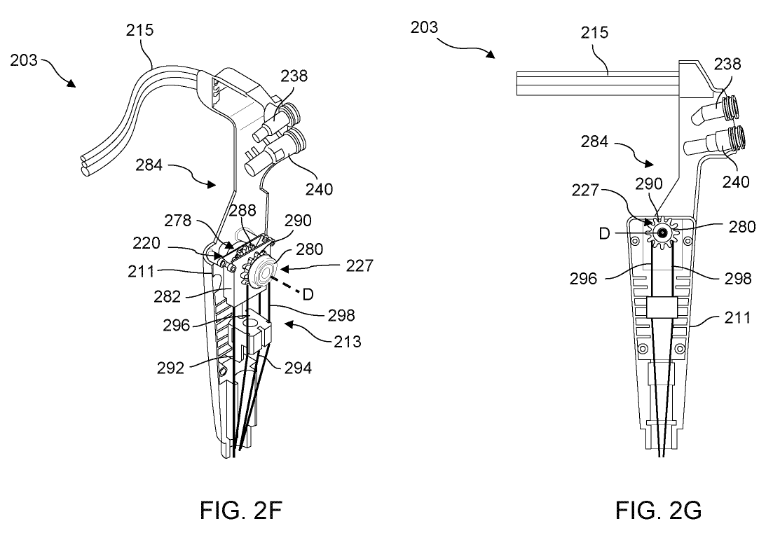

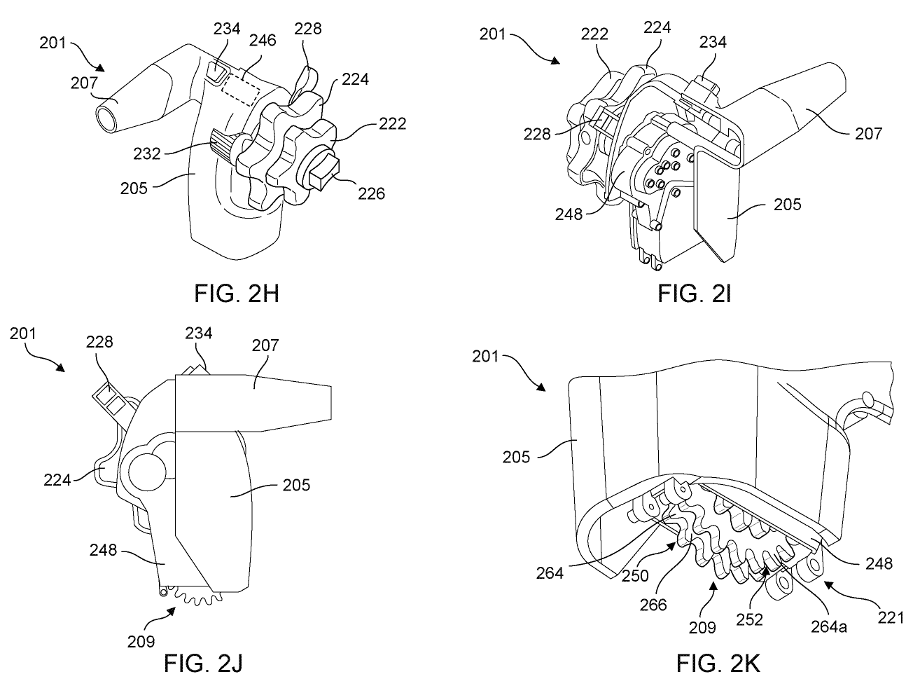

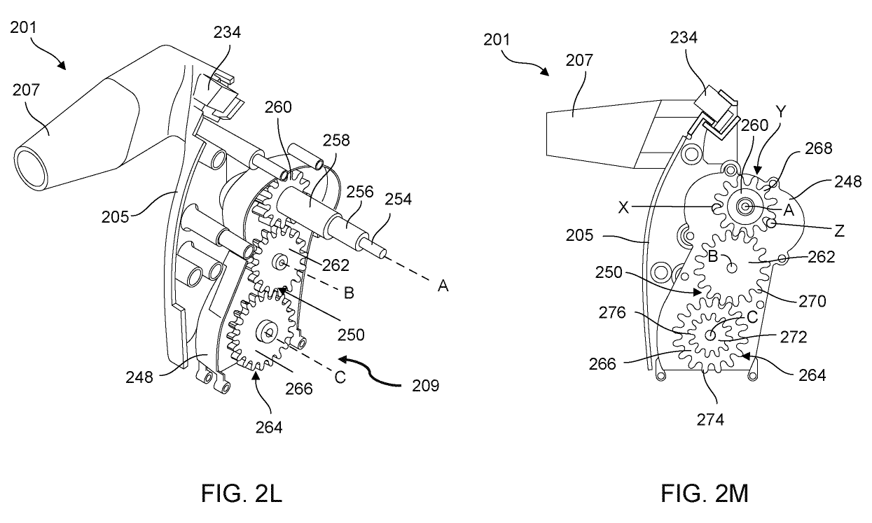

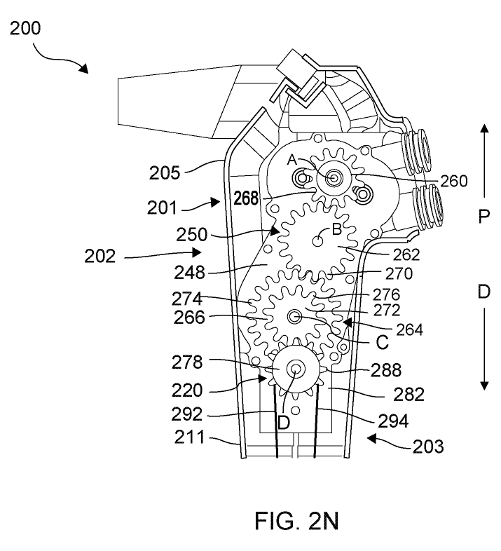

FIGS. 2A-2N illustrate an exemplary medical device 200 that includes a reusable portion 201 (e.g., a first portion) removably coupled to a disposable portion 203 (e.g., a second portion). FIG. 2A illustrates medical device 200 with reusable portion 201 partially detached from disposable portion 203. FIGS. 2B-2G illustrate features of disposable portion 203, and FIGS. 2H-2M illustrate features of reusable portion 201. FIG. 2N illustrates internal components of a handle 202 of medical device 200 that is formed when reusable portion 201 is coupled to disposable portion 203.

As shown in FIG. 2A, disposable portion 203 may include a housing 211 and an insertion portion 204 extending distally from housing 211. Housing 211 and the components therein may form a portion of handle 202. Insertion portion 204 may include any of the features of insertion portion 104, unless otherwise specified herein. Insertion portion 204 may include a shaft 206 similar to shaft 106, and a distal tip 208 similar to distal tip 108. For example, shaft 206 may include a steerable section 216 similar to steerable section 116. Distal tip 208 may include an imaging device 210 similar to imaging device 110, a light source 212 similar to light source 112, and an elevator 214 similar to elevator 114.

Disposable portion 203 may include one or more working channels extending through shaft 206, and one or more ports 230 in communication with the working channel(s). Port(s) 230 may extend from housing 211. Disposable portion 203 may include an umbilicus 215 extending from housing 211. Umbilicus 215 may only include tubes, conduits, or other components necessary for supplying fluid (e.g., air or water) or suction to medical device 200. Umbilicus 215 may not include any wires or cables for communication with electrical components. Disposable portion 203 may include a first valve well 238 for receiving a suction valve and a second valve well 240 for receiving an air/water valve. Umbilicus 215 may connect medical device 200 to a source of fluid and a source of suction, and valves inserted into first and second valve wells 238, 240 may be used to control delivery of a fluid (air or water, for example) through tubing extending through shaft 206 and application of suction through the working channel(s), respectively.

Reusable portion 201 may include a housing 205 and a gear mechanism 209 disposed within housing 205. When housing 205 is combined with housing 211, a housing of handle 202 may be formed. Reusable portion 201 may include a first control knob 222 (e.g., a left/right knob) and a second control knob 224 (e.g., an up/down knob), each being coupled to gear mechanism 209. Gear mechanism 209 may be configured to interact or engage with a pull wire mechanism 213 (described in further detail below) of disposable portion 203 to deflect steerable section 216 of shaft 206. Accordingly, when reusable portion 201 is attached to disposable portion 203, first and second control knobs 222, 224 may be indirectly coupled to pull wire mechanism 213 to control steerable section 216 of shaft 206. First control knob 222 may provide left/right control of steerable section 216, and second control knob 224 may provide up/down control of steerable section 216. Reusable portion 201 may include a first locking mechanism 226 and a second locking mechanism 228 (e.g., knobs or levers) for locking first and second control knobs 222, 224 in a desired position, respectively. Reusable portion 201 may include a control lever 232 (FIG. 2H) for raising or lowering elevator 214.

Reusable portion 201 may include an umbilicus 207 extending from housing 205. Umbilicus 207 may only include electrical wires, cables, or other electrical components necessary for transmitting signals, receiving signals, or delivering power to medical device 200. Umbilicus 207 may not include tubing for transmitting air, water, or suction. For example, umbilicus 207 may connect medical device 200 to a control unit, an imaging system, a display, or power supply for, e.g., controlling imaging device 210 or light source 212, displaying images captured by imaging device 210 on a display, or powering components of medical device 200.

As shown in FIG. 2H, reusable portion 201 may include a printed circuit board (PCB) 246 within housing 205 and that is electrically coupled to umbilicus 207. PCB 245 may be coupled to or may include an electrical connector. For example, PCB 246 may be proximate a proximalmost end of housing 205. As shown in FIG. 2B, disposable portion 203 may include a decoder board 286 (which may be a PCB) within housing 211 and that is electrically coupled to imaging device 210. For example, decoder board 286 may be within a distal portion of housing 211 or within housing 211 proximate a distalmost end of housing 211, which may help improve signal quality by reducing a length of wire(s) extending from imaging device 210 to decoder board 286. Decoder board 286 may be configured to transmit signals from imaging device 210 to PCB 246, and PCB 246 may transmit the signals to umbilicus 207. As shown in FIGS. 2H-2J, 2L, and 2M, reusable portion 201 may include an image capture button 234 configured to capture video or still images from imaging device 210. PCB 246 may be connected to image capture button 234. PCB 246 may be used only to receive signals from image capture button 234 and to transmit data between the external controller and decoder board 286. In other words, PCB 246 may not include any camera-specific hardware or software. Decoder board 286 may include camera-specific hardware or software. Thus, reusable portion 201 may be used with a variety of types of disposable portions 203, which may have different types of cameras.

Housing 205 of reusable portion 201 may have a shape complementary to a shape of housing 211 of disposable portion 203 to facilitate forming handle 202 of medical device 200. For example, as shown in FIGS. 2A and 2B-2G, a proximal portion of housing 211 may define a cavity 284 that is sized and shaped to receive reusable portion 201. Housings 205 and 211 may each include corresponding connecting elements that may connect to form handle 202. For example, a mechanical latch may be used to fix housing 205 to housing 211. When housings 205 and 211 are connected, housings 205 and 211 may form or define handle 202 that extends from a proximalmost end 242 to a distalmost end 244. For example, housing 211 may extend from proximalmost end 242 of handle 202 to distalmost end 244 of handle 202, while housing 205 may extend along a proximal portion of handle 202 (e.g., from a midpoint of handle 202 to proximalmost end 242).

As mentioned above, disposable portion 203 may include pull wire mechanism 213 that is distal to cavity 284. A proximalmost portion of pull wire mechanism 213 may extend into cavity 284, in some aspects. Referring to FIG. 2E, pull wire mechanism 213 may include a first spool assembly 220 and a second spool assembly 227. First spool assembly 220 may include a first spool 278 that is coupled to a first gear 288. Second spool assembly 227 may include a second spool 280 that is coupled to a second gear 290. In aspects, first spool 278 may be rotationally fixed relative to first gear 288, such that rotation of first gear 288 causes corresponding rotation of first spool 278. Similarly, second spool 280 may be rotationally fixed relative to second gear 290, such that rotation of second gear 290 causes corresponding rotation of second spool 280. In other words, an entirety of first spool assembly 220 may be rotatable together, and an entirety of second spool assembly 227 may be rotatable together. First and second spool assemblies 220, 227 may be disposed adjacent to one another and in a casing 282 that is within housing 211. First and second spool assemblies 220, 227 may be disposed within respective spaces of casing 282, and first and second gears 288, 290 may be partially exposed from casing 282.

In some aspects, casing 282 may include a first wall 283a, a second wall 283b, and a third wall 283c. First wall 283a may be on a first side of pull wire mechanism 213, adjacent to first spool assembly 220 and separating pull wire mechanism 213 from other portions of a cavity 285 defined by housing 211 (e.g., in a distal portion of housing 211). First wall 283a may define a recess for receiving first spool 278. Second wall 283b may be on a second side of pull wire mechanism 213, adjacent to second spool assembly 227 and adjacent to a wall of housing 211. Third wall 283c may be between first spool assembly 220 and second spool assembly 227. For example, third wall 283c may be adjacent to and between first gear 288 and second gear 290. First and second spools 278, 280 may be positioned on opposite, outer sides of pull wire mechanism 213, with first and second gears 288, 290 closer to a center of pull wire mechanism 213. In some examples, third wall 283c may be a thin, flexible sheet of low friction material that may be held in place by first and second walls 283a, 283b.

FIGS. 2F and 2G illustrate a portion of housing 211 and casing 282 removed or transparent to expose further features of pull wire mechanism 213. As shown, first and second spool assemblies 220, 227 may each be coupled to two pull wires that extend distally to steerable section 216 of shaft 206. First spool assembly 220 may be coupled to a first pull wire 292 and a second pull wire 294. For example, first pull wire 292 and second pull wire 294 may each be coupled to (e.g., wrap around) first spool 278. Second spool assembly 227 may be coupled to a third pull wire 296 and a fourth pull wire 298. For example, third pull wire 296 and fourth pull wire 298 may be coupled to (e.g., wrap around) second spool 280. First and second spool assemblies 220, 227 may each be configured to rotate about a rotation axis D (e.g., coaxially) independently of one another to move pull wires 292, 294 and pull wires 296, 298, respectively, to deflect steerable section 216 of shaft 206. First and second spool assemblies 220, 227 may each be configured to interact or engage with gear mechanism 209 of reusable portion 201 via first and second gears 288, 290, respectively, as described in further detail below.

Referring to FIGS. 2I-2M, gear mechanism 209 may be disposed in a casing 248 within housing 205 of reusable portion 201. As shown in FIG. 2K, gear mechanism 209 may include a first gear assembly 250 (e.g., a first gear train) and second gear assembly 252 (e.g., a second gear train). First gear assembly 250 may be rotatably coupled to or engaged with second control knob 224, and second gear assembly 252 may be rotatably coupled to or engaged with first control knob 222. First gear assembly 250 may be configured to engage with first spool assembly 220 of pull wire mechanism 213, and second gear assembly 252 may be configured to engage with second spool assembly 227 of pull wire mechanism 213. As will be discussed in greater detail below, first gear assembly 250 may include a third gear 264, and second gear assembly 252 may include a third gear 264a. As more clearly shown in FIGS. 2J and 2K, third gears 264, 264a may be partially exposed from casing 248. For example, third gears 264, 264a may protrude distally from a distalmost end of casing 248 and a distalmost end of housing 205. Third gears 264, 264a may be configured to interact or engage with first and second gears 288, 290 of first and second spool assemblies 220, 227, respectively. For example, external teeth of third gear 264 may engage with (e.g., mesh with) external teeth of first gear 288, and external teeth of third gear 264a may engage with (e.g., mesh with) external teeth of second gear 290.

Second gear assembly 252 may be adjacent to first gear assembly 250, and may include the same number of gears, types of gears, and arrangement of gears as first gear assembly 250 (e.g., to provide for a same amount of torque to be applied to produce left/down or up/right movement). Alternatively, first and second gear assemblies 250, 252 may differ from one another (e.g., to require greater torque to be applied for left/right or up/down movement). Additionally, casing 248 or another portion of housing 205 may include one or more connecting elements 221 that correspond to one or more connecting elements 223 of casing 282 (FIG. 2E) to connect casing 248 to casing 282.

FIGS. 2L and 2M illustrate a portion of housing 205 removed, as well as first and second control knobs 222, 224, first and second locking mechanisms 226, 228, and control lever 232 removed, to expose further features of reusable portion 201, including gear mechanism 209 and casing 248. Second gear assembly 252 is also removed from FIGS. 2L and 2M to more clearly show first gear assembly 250. Second gear assembly 252 may have any property of first gear assembly 250, except as specified herein.

Reusable portion 201 may include a first shaft 254 (e.g., a mounting shaft) that is fixedly coupled to casing 248 at a mounting location Y, a second shaft 256 that surrounds first shaft 254, and a third shaft 258 that surrounds second shaft 256. Second shaft 256 may be coupled to first control knob 222 (e.g., rotationally fixed to first control knob 222), and third shaft 258 may be coupled to second control knob 224 (e.g., rotationally fixed to second control knob 224). First shaft 254, second shaft 256, and third shaft 258 may be concentric. Second and third shafts 256, 258 may rotate about a rotation axis A independently of one another (with rotation of first control knob 222 and second control knob 224, respectively) and relative to first shaft 254. For example, rotation of first control knob 222 about rotation axis A may rotate second shaft 256 about rotation axis A, but may not rotate third shaft 258 (and second control knob 224) about rotation axis A. Similarly, rotation of second control knob 224 about rotation axis A may rotate third shaft 258 about rotation axis A, but may not rotate second shaft 256 (and first control knob 222) about rotation axis A.

Referring to FIGS. 2L and 2M, first gear assembly 250 that is coupled to second control knob 224 may include a first gear 260 with external teeth 268, a second gear 262 (e.g., an idler gear) with external teeth 270, and third gear 264. First gear 260 may be a proximalmost of the gears, with third gear 264 being the distalmost of the gears of gear assembly 250. Second gear 262 may be a middle gear between first gear 260 and third gear 264. Third gear 264 may be a compound gear including a large gear 266 (outer gear) with external teeth 274, and a small gear 272 (inner gear) with external teeth 276, wherein large gear 266 and small gear 272 share an axle. First gear 260 may be coupled to second shaft 256 such that rotation of second shaft 256 about rotation axis A rotates first gear 260 about rotation axis A. For example, first gear 260 may be fixedly coupled (e.g., rotationally fixedly coupled) to second shaft 256. Second gear 262 may be positioned between first gear 260 and third gear 264, such that external teeth 270 of second gear 262 is engaged with external teeth 268 of first gear 260 and external teeth 274 of large gear 266. When first and second control knobs 222, 224 are mounted at a location Y, shown in FIG. 2M, first gear 260 may be aligned with second and third gears 262, 264, such that a straight line extends through rotation axes A, B, and C of first gear 260, second gear 262, and third gear 264, respectively. Third gear 264 may be configured to engage with first spool assembly 220, as discussed above, to deflect steerable section 216 of shaft 206 in the up or down direction.

Although not shown in FIGS. 2L and 2M, second gear assembly 252 that is coupled to first control knob 222 may include the same number, the same types, and the same arrangement of gears as first gear assembly 250. For example, second gear assembly 252 may include a first gear adjacent to and similar to first gear 260, except the first gear of second gear assembly 252 is coupled to third shaft 258. The first gear of second gear assembly 252 may be coupled to third shaft 258 such that rotation of third shaft 258 about rotation axis A rotates the first gear of second gear assembly 252 about rotation axis A. Second gear assembly 252 may include a second gear adjacent to and similar to second gear 262, and a third gear 264a (see FIG. 2K) similar to and adjacent to third gear 264. Third gear 264a may engage with second spool assembly 227 to deflect steerable section 216 of shaft 206 in the left or right direction. In an alternative, third gear 264a of second gear assembly 252 may be different from third gear 264 of first gear assembly 250.

Referring to FIG. 2M, casing 248 may provide two other mounting locations X and Z for mounting first and second control knobs 222, 224 (and, thus, first gear 260) on casing 248, and thus may provide two additional rotation axes for first and second control knobs 222, 224. At mounting locations X and Z, first gear 260 may be offset from second and third gears 262, 264, such that a straight line does not extend through rotational axes A, B, and C. Mounting locations X, Y, and Z may be arranged along an arc. A cavity defined by casing 282 may be shaped so as to accommodate gears mounted at locations X, Y, and Z. In other words, a cavity defined by casing 282 may be wider at a proximal end. Alternatively, mounting locations X, Y, and Z may be arranged along a straight line. For example, in this alternative, a size of second gear 262 and a rotation axis of second gear 262 may be modified to facilitate arranging mounting locations X, Y, and Z in a straight line.

Medical device 200 in use will now be described with reference to FIG. 2N, which shows reusable portion 201 coupled to disposable portion 203 to form handle 202. Second gear assembly 252 and second spool assembly 227 are removed from FIG. 2N to more clearly show first gear assembly 250 engaged with first spool assembly 220.

In use, first gear assembly 250 may provide a mechanism for second control knob 224 to rotate second shaft 256 about rotation axis A in order to move first and second pull wires 292, 294 (e.g., proximally or distally) to deflect steerable section 216 of shaft 206 in the up or down direction. When a user rotates second control knob 224 clockwise about rotation axis A, second shaft 256 and first gear 260 also rotate clockwise about rotation axis A, and external teeth 268 of first gear 260 engage (e.g., mesh with) external teeth 270 of second gear 262 to rotate second gear 262 counterclockwise about rotation axis B. When second gear 262 rotates counterclockwise, external teeth 270 of second gear 262 engage (e.g., mesh with) external teeth 274 of large gear 266 to rotate large gear 266 clockwise about rotation axis C. When large gear 266 rotates clockwise about rotation axis C, small gear 272 also rotates clockwise about rotation axis C. When small gear 272 rotates clockwise, external teeth 276 of small gear 272 engage with (e.g., mesh with) external teeth of first gear 288 of spool assembly 220 to rotate spool assembly 220 (first gear 288 and first spool 278) counterclockwise about rotation axis D to move first and second pull wires 292, 294. Rotation axis D of first gear 288 of spool assembly 220 may be parallel to but not coaxial with rotation axis C of third gear 264. First gear 288 of spool assembly 220 may be at least partially distal to third gear 264. Movement of first and second pull wires 292, 294 may deflect steerable section 216 of shaft 206 in the up or down direction (or a left or right direction).

Second gear assembly 252 may provide a mechanism for first control knob 222 to rotate third shaft 258 about rotation axis A in order to move third and fourth pull wires 296, 298 (e.g., proximally or distally) to deflect steerable section 216 of shaft 206 in the left or right direction. It will be appreciated that second gear assembly 252 may engage with second spool assembly 227 in the same manner described above for first gear assembly 250 and first spool assembly 220.

After use, reusable portion 201 may be detached from disposable portion 203, and attached to another disposable portion 203 for another use (e.g., another medical procedure). It will be appreciated that a length or a diameter of shaft 206 may vary, and a diameter or an angulation of steerable section 216 may also vary. Additionally, a type or a number of imaging devices 210 may also vary. Varying these features of disposable portion 203 may allow an operator to perform different procedures with the same reusable portion 201.

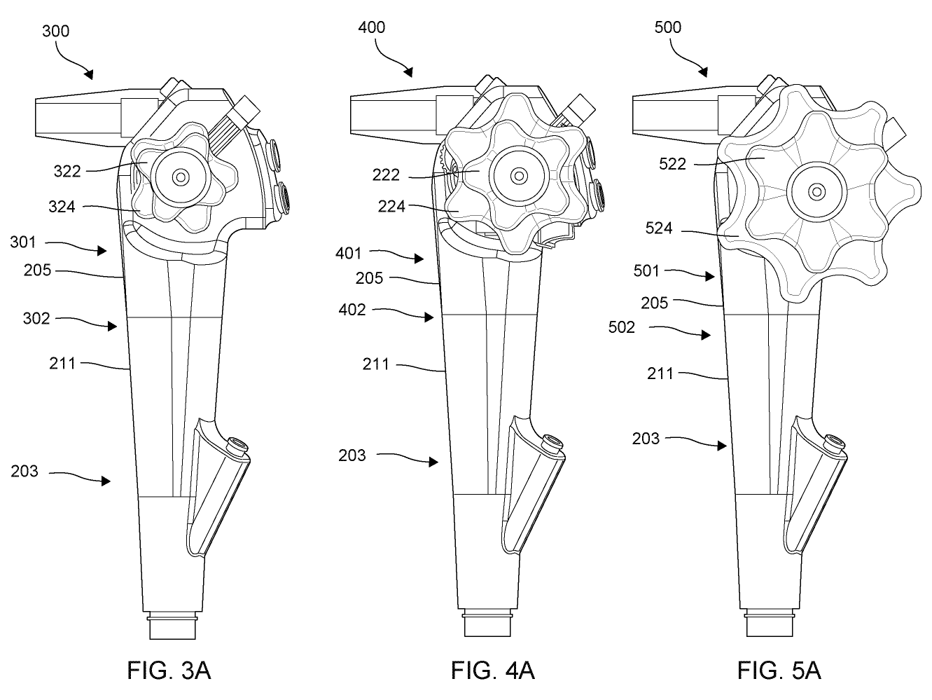

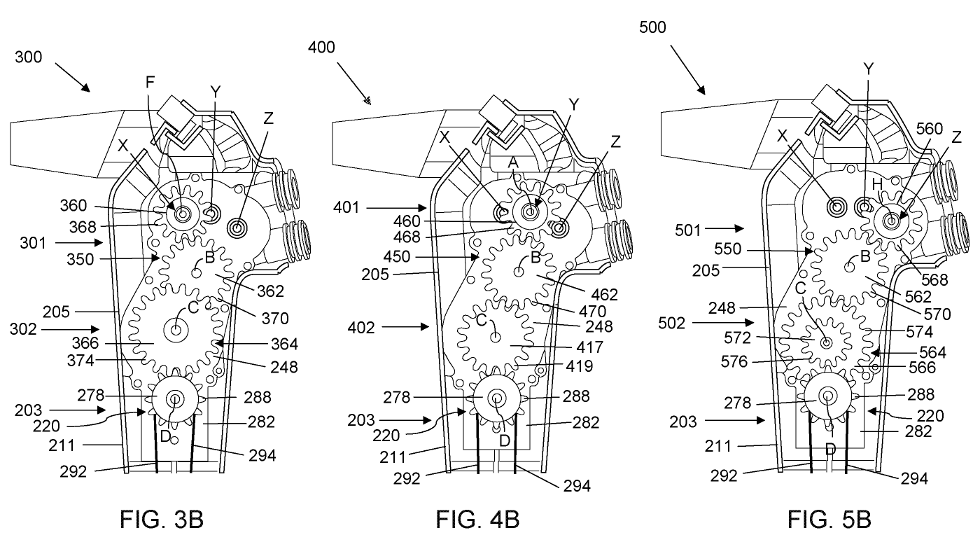

As mentioned above, features of the control knobs and the gear assemblies may vary. This is further illustrated in FIGS. 3A-3B, 4A-4B, and 5A-5B. For example, as shown in FIGS. 3A, 4A, and 5A, the control knobs may vary in size (e.g., small, medium, or large). For example, as shown in FIGS. 3A-3B, 4A-4B, and 5A-5B, the positioning of the control knobs on the reusable portion may vary, and thus the positioning of the first gear of the gear assemblies may vary.

Additionally, the third gear of the gear assemblies may vary in type, size, or arrangement to provide different gear ratios, e.g., high, low, or 1:1. The different arrangements disclosed herein may improve operator ergonomics and may be customized to a particular user to enhance usability and to reduce strain on an operator. For example, a low gear ratio (e.g., between gear 262 (or its corollaries discussed below) and a portion of gear 264 (or its corollaries discussed below) that engages gear 262) may require an operator to exert less force on the control knobs as compared to a 1:1 gear ratio to achieve a same amount of articulation or deflection of steerable section 216, while a high gear ratio may require an operator to exert more force on the control knobs as compared to a 1:1 gear ratio to achieve a same amount of articulation or deflection of steerable section 216. Reducing the forces required to rotate the control knobs (as provided by a low gear ratio) or reducing the amount of movement needed to rotate the control knobs (as provided by a high gear ratio) may help relieve injuries related to muscle fatigue. Features of the aspects disclosed herein may be combined in any suitable manner. For example, different size knobs may be combined with different gear ratios. The arrangements shown herein are merely exemplary.

FIGS. 3A and 3B illustrate features of an exemplary medical device 300 including a reusable portion 301 and disposable portion 203. FIG. 3A illustrates an external view of a handle 302 of medical device 300, and FIG. 3B illustrates internal components of handle 302 of medical device 300. Reusable portion 301 may include any of the features of reusable portion 201, unless otherwise specified herein.

In this example, reusable portion 301 may include a first control knob 322 and a second control knob 324, each having a size smaller than first and second control knobs 222, 224, respectively. For example, first and second control knobs 322, 324 may each have a diameter less than a diameter of first and second control knobs 222, 224, respectively. For example, first and second control knobs 322, 324 may be a set of small-sized control knobs, and first and second control knobs 222, 224 may be a set of medium-sized control knobs. Additionally, first and second control knobs 322, 324 may be mounted on casing 248 at location X, and may rotate about a rotation axis F (FIG. 3B).

Referring to FIG. 3B, reusable portion 301 may include a first gear assembly 350 that is coupled to second control knob 324. First gear assembly 350 may be configured to engage with first spool assembly 220 of disposable portion 203 to deflect steerable section 216 of shaft 206 in the up or down direction.

First gear assembly 350 may include a first gear 360 with external teeth 368, a second gear 362 (e.g., an idler gear) with external teeth 370, and a third gear 364. First gear 360 may be similar to first gear 260 except first gear 360 is positioned at location X offset from second and third gears 362, 364, such that a straight line does not extend through axes F, B, and C. Because first gear 360 is rotatably fixedly coupled to second control knob 324, first gear 360 also rotates about rotation axis F when second control knob 324 rotates about rotation axis F. Second gear 362 may be similar to second gear 262. Similar to third gear 264, third gear 364 may be a compound gear including a large gear 366 with external teeth 374, and a small gear with external teeth that is behind large gear 366 in FIG. 3B. In this example, external teeth 374 of large gear 366 may be configured to engage the external teeth of first gear 288 of first spool assembly 220, while the external teeth of the small gear may be configured to engage external teeth 370 of second gear 362.

The arrangement of third gear 364 may provide an operator with a high gear ratio, e.g., a gear ratio of 20:12. During use, a high gear ratio may result in faster articulation or deflection of steerable section 216 of shaft 206, but may require an operator to exert more force on second control knob 324 to rotate second control knob 324. For example, about 66.7% greater operator hand force as compared to a gear assembly 450 having a 1:1 gear ratio (discussed below).

Although not illustrated, reusable portion 301 may include a second gear assembly similar to first gear assembly 350, except coupled to first control knob 322 and configured to couple to second spool assembly 227 for deflecting steerable section 216 of shaft 206 in the left or right direction.

FIGS. 4A and 4B illustrate features of an exemplary medical device 400 including a reusable portion 401 and disposable portion 203. FIG. 4A illustrates an external view of a handle 402 of medical device 400, and FIG. 4B illustrates internal components of handle 402 of medical device 400. Reusable portion 401 is similar to reusable portion 201, except for the third gear of the first and second gear assemblies.

Referring to FIG. 4B, reusable portion 401 may include a first gear assembly 450 that is coupled to second control knob 224. First gear assembly 450 may be configured to engage with first spool assembly 220 of disposable portion 203 to deflect steerable section 216 of shaft 206 in the up or down direction. First gear assembly 450 may include a first gear 460 with external teeth 468 similar to first gear 260 with external teeth 268, and a second gear 462 (e.g., an idler gear) with external teeth 470 similar to second gear 262 with external teeth 270. First gear assembly 450 may include a third gear 417 that is a single gear instead of a compound gear. In this example, external teeth 419 of third gear 417 may be configured to engage the external teeth of first gear 288 of first spool assembly 220 and external teeth 470 of second gear 462. Third gear 417 may have a smaller diameter than large gear 266 and a larger diameter than small gear 272. Third gear 417 may provide an operator with a 1:1 gear ratio, e.g., a gear ratio of 16:16.

Although not illustrated, disposable portion 401 may include a second gear assembly similar to first gear assembly 450, except coupled to first control knob 422 and configured to couple to second spool assembly 227 for deflecting steerable section 216 of shaft 206 in the left or right direction.

FIGS. 5A and 5B illustrate features of an exemplary medical device 500 including a reusable portion 501 and disposable portion 203. FIG. 5A illustrates an external view of a handle 502 of medical device 500, and FIG. 5B illustrates internal components of handle 502 of medical device 500. Reusable portion 501 may include any of the features of reusable portion 201, unless otherwise specified herein.

In this example, reusable portion 501 may include a first control knob 522 and a second control knob 524, each having a size larger than first control knobs 222, 322, and second control knobs 224, 324, respectively. For example, first and second control knobs 522, 524 may each have a diameter greater than a diameter of first control knobs 222, 322, and second control knobs 224, 324, respectively. For example, first and second control knobs 522, 524 may be a set of large-sized control knobs. Additionally, first and second control knobs 522, 524 may be mounted on casing 248 at location Z, and may rotate about a rotation axis H (FIG. 5B).

Referring to FIG. 5B, reusable portion 501 may include a first gear assembly 550 that is coupled to second control knob 524. First gear assembly 550 may be configured to engage with first spool assembly 220 of disposable portion 203 to deflect steerable section 216 of shaft 206 in the up or down direction. First gear assembly 550 is similar to first gear assembly 250, except for the position of the first gear. First gear assembly made include a second gear 562 with external teeth 570 similar to first gear 262 with external teeth 270, and may include a third gear 564 similar to third gear 264. For example, third gear 564 may be a compound gear including a large gear 566 with external teeth 574, and a small gear 572 with external teeth 576. This arrangement of third gear 564 (and third gear 264) may provide an operator with a low gear ratio, e.g., a gear ratio of 12:20. During use, a low gear ratio may result in slower articulation or deflection of steerable section 216 of shaft 206, but may require an operator to exert less force on second control knob 524 to rotate second control knob 524. For example, about 40% less operator hand force as compared to gear assembly 450.

In this example, first gear 560 and second control knob 524 are positioned at location Z offset from second and third gears 562, 564, such that they are rotatable about a rotation axis H. Because first gear 560 is coupled to second control knob 524, first gear 560 also rotates about rotation axis H when second control knob 524 rotates about rotation axis H. A straight line may not extend through axes H, B, and C.

Although not illustrated, disposable portion 501 may include a second gear assembly similar to first gear assembly 550, except coupled to first control knob 522 and configured to couple to second spool assembly 227 for deflecting steerable section 216 of shaft 206 in the left or right direction.

It will be appreciated that features of reusable portions 201, 301, 401, 501 may be combined in any combination or subcombination. In at least one example, gear assembly 250 may include third gear 364 in place of third gear 264. In another example, gear assembly 250 may include third gear 417 in place of third gear 264.

It will be appreciated that features of reusable portions 201, 301, 401, 501 may be modified based on an operator’s control knob size preference (e.g., small, medium, or large) or gear ratio preference (e.g., high gear ratio, low gear ratio, 1:1 gear ratio). For example, an operator may select third gear 417 for a 1 to 1 gear ratio. In other examples, an operator may select third gear 264, 364 or 564 to adjust the gear ratio, e.g., increase or decrease the gear ratio. In some examples, control knobs 222, 224, 322, 324, 522, 524 may vary in color. For example, different colors may be used to correspond to different sizes (e.g., small, medium, large) of control knobs 222, 224, 322, 324, 522, 524.

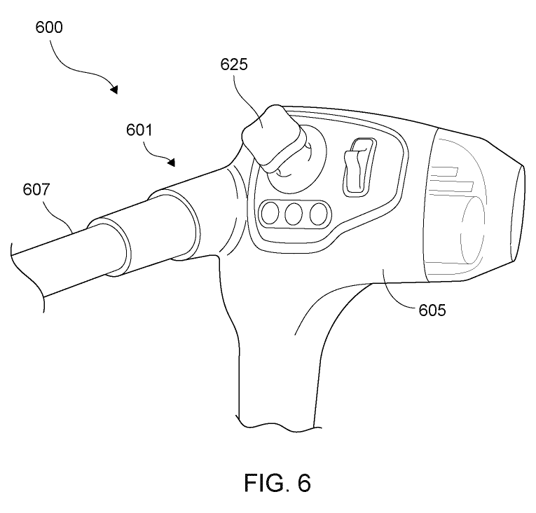

An exemplary motorized medical device 600 including a reusable portion 601 and disposable portion 203 will now be described with reference to FIG. 6. Medical device 600 may have any of the properties of the medical devices referenced in Provisional Application No. 63/798,639, filed on May 2, 2025, and incorporated by reference in its entirety herein. FIG. 6 illustrates reusable portion 601. Reusable portion 601 may include any of the features of reusable portions 201, 301, 401, 501. For example, reusable portion 601 may include an umbilicus 607 similar to umbilicus 207, and a housing 605 similar to housing 205.

In this example, reusable portion 601 may include a joystick 625 instead of control knobs for controlling deflection of steerable section 216 of shaft 206. Joystick 625 may be coupled to a first motor and a second motor within housing 605. The first and second motors may be coupled to a first gear assembly and a second gear assembly within housing 605, respectively, and the first and second gear assemblies may be configured to couple to first and second spool assemblies 220, 227 of disposable portion 203. Accordingly, the first and second motors may be used to drive deflection of steerable section 216 of shaft 206 in the left, right, up, or down direction.

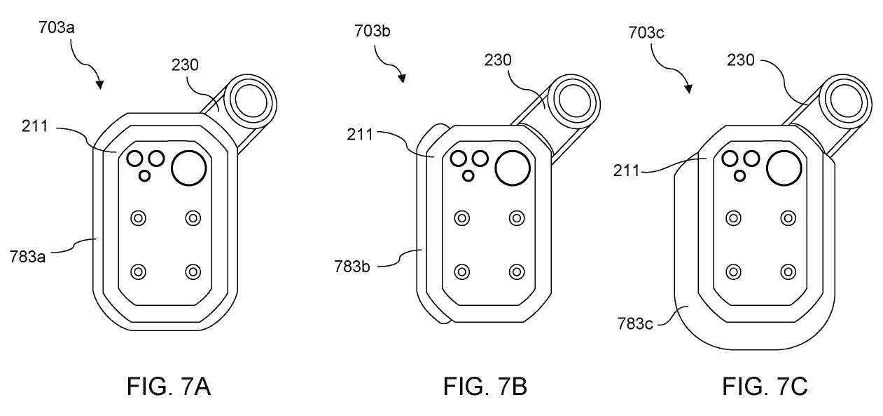

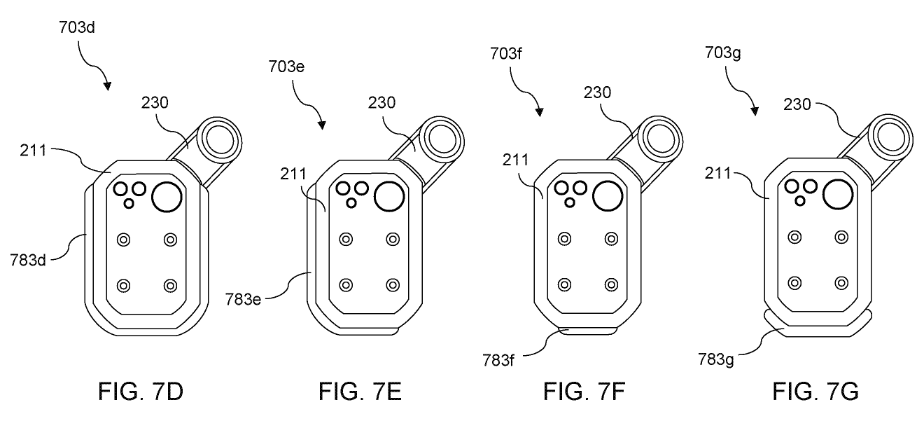

Additionally, or optionally, disposable portion 203 may include a grip portion to provide a user with different handle sizes or grip sizes, e.g., when disposable portion 203 is coupled to a respective reusable portion 201, 301, 401, 501, 601. The gripping portions herein may be configured to partially or completely surround or cover a circumference of disposable portion 203, e.g., partially or completely surround or cover a circumference of a distal portion of disposable portion 203. For example, the gripping portions herein may be configured to partially or completely surround or cover exterior surfaces of housing 211 of disposable portion 203. Various exemplary gripping portions are illustrated in FIGS. 7A-7G.

FIG. 7A illustrates a cross-sectional view of a gripping portion 783a completely surrounding or covering a circumference of housing 211 of a disposable portion 703a. FIG. 7B illustrates a cross-sectional view of a gripping portion 783b partially surrounding or covering a circumference of housing 211 of a disposable portion 703b. FIG. 7C illustrates a cross-sectional view of a gripping portion 783c partially surrounding or covering a circumference of housing 211 of a disposable portion 703c. Gripping portion 783c may have a thickness or a width greater than a thickness or a width of gripping portions 783a, 783b. FIG. 7D shows a cross-sectional view of a disposable portion 703d with a gripping portion 783d surrounding approximately 75% of housing 211. FIG. 7E shows a cross-sectional view of a disposable portion 703e with a gripping portion 783e surrounding approximately half of housing 211. As shown, gripping portion 783e may cover two adjacent sides of housing 211. FIG. 7F shows a disposable portion 703f having a gripping portion 783f covering one side of housing 211. FIG. 7G shows a disposable portion 703g having a gripping portion 783g covering one side of housing 211. As compared with disposable portion 703f having gripping portion 783f, gripping portion 783g may cover more of housing 211. For example, gripping portion 783g may extend along a transition portion between adjacent sides of housing 211.

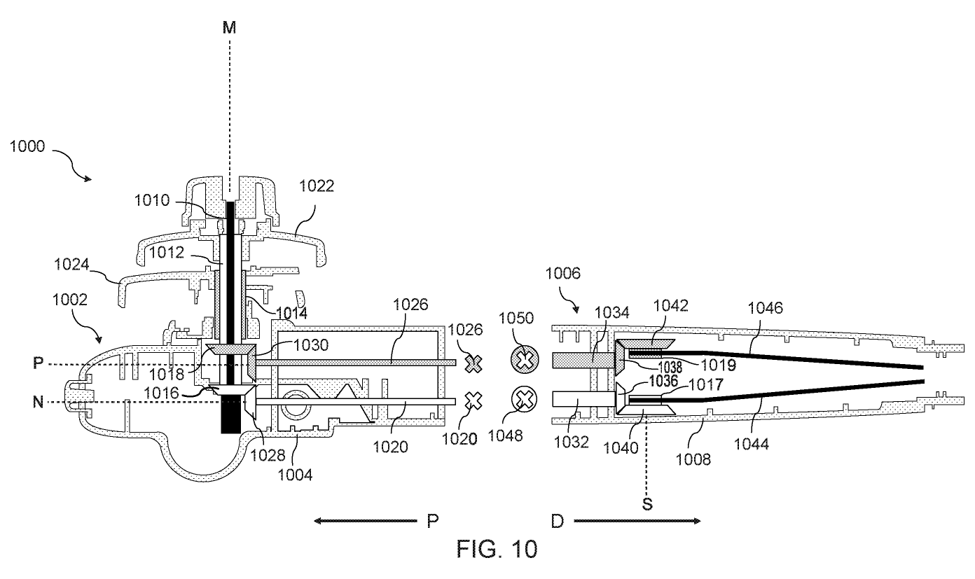

The gripping portions shown in FIGS. 7A-7G may be utilized to change an overall outside dimension of the disposable portions, while still allowing for compatibility with the reusable portions. An operator may be able to select a desired gripping portion and apply the gripping portion to a disposable portion. The gripping portion may provide a configuration of a disposable portion that suits his or her preferences at a given time. For example, disposable portions 703a-703g may have a greater width than disposable portion 203 lacking a gripping portion. Disposable portion 703c may be wider in a horizontal direction across the page of the drawings than disposable portion 703a, which may be wider than disposable portion 703b. The gripping portions may be placed in particular locations to provide for particular ergonomic benefits. For example, disposable portion 703c may be thicker (in an up/down direction on the page) than disposable portion 703b. Disposable portion 703c may also be thicker than disposable portion 703a, but disposable portion 703a may have gripping portion 783a on all sides, while disposable portion 703c may lack gripping portion 783c on one side. The arrangements of the gripping portions are merely exemplary. Gripping portions 783a-783g may also be used in combination with any of the disposable portions of the medical devices of FIGS. 10-16 described below.

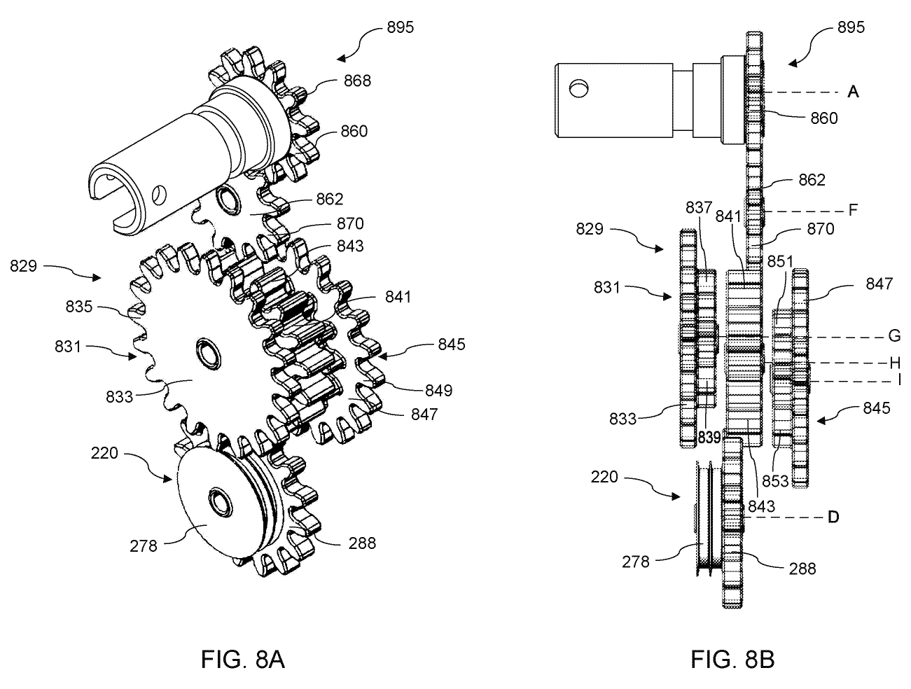

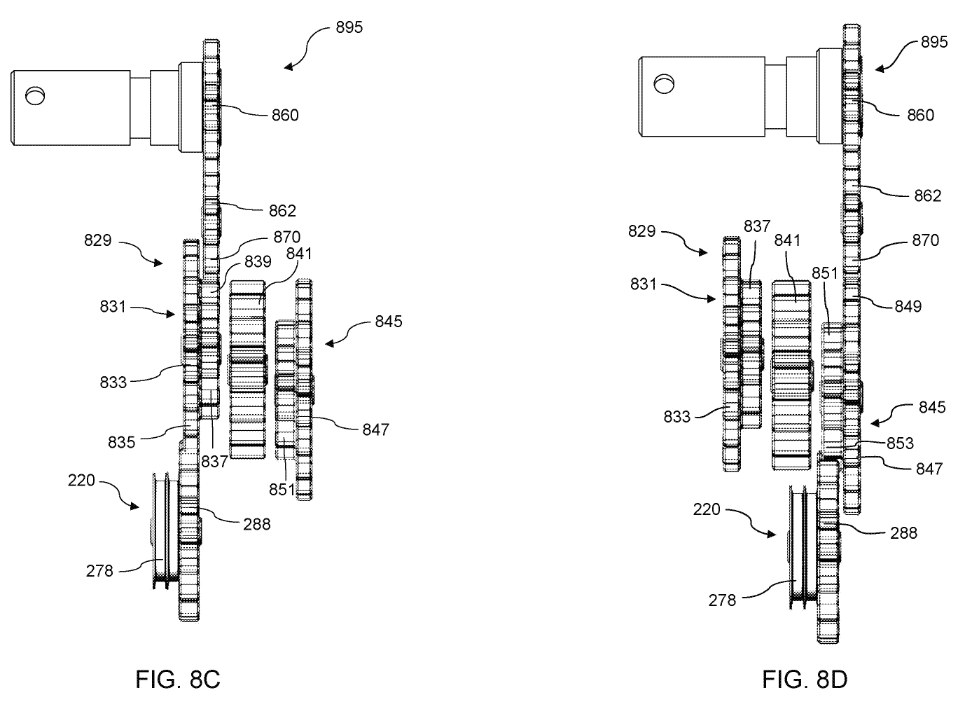

FIGS. 8A-8D illustrate an exemplary gear assembly 895 that may allow an operator to change gear ratios during or prior to a medical procedure, e.g., between a high gear ratio, a low gear ratio, or a 1:1 gear ratio (e.g., a neutral gear ratio). Gear assembly 895 may be used in place of any one of the first and second gear assemblies described above, e.g., in place of any one of gear assemblies 250, 252, 350, 450, 550. For ease of discussion, gear assembly 895 will be described in place of first gear assembly 250 of medical device 200. It will be appreciated that gear assembly 895 may also be used with any other of the gear assemblies described herein. A medical device may include two gear assemblies 895, one for each knob of the medical device.

Referring to FIG. 8A, gear assembly 895 may include a first gear 860 with external teeth 868, a second gear 862 (e.g., an idler gear) with external teeth 870, and a gear unit 829. First gear 860 may be rotatably fixedly coupled to second control knob 224 or any other control knob described herein. Second gear 862 may be disposed axially (e.g., in a direction substantially parallel to a longitudinal axis of handle 202 of medical device 200) between first gear 860 and gear unit 829. Gear unit 829 may be engageable (e.g., removably engageable) with first spool assembly 220 or any other spool assembly referenced herein. For example, as shown in FIGS. 8A-8D, gear unit 829 may be disposed axially between second gear 862 and first spool assembly 220 when reusable portion 201 is coupled to disposable portion 203.

Gear unit 829 may include a first compound gear 831, a second compound gear 845, and a single gear 841. In aspects, compound gears 831, 845 and single gear 841 may be connected to one another. Single gear 841 may be disposed between first and second compound gears 831, 845, in a direction of a rotation axis extending through any of the gears 831, 841, 845. First compound gear 831 may include a large gear 833 and a small gear b(see FIGS. 8B-8D), and second compound gear 845 may include a large gear 847 and a small gear 851 (see FIGS. 8B-8D). First and second compound gears 831, 845 may be positioned on opposite sides of single gear 841, with small gears 837, 851 closer to single gear 841. Compound gears 831, 845 and single gear 841 may move laterally (e.g., move in a direction transverse to a longitudinal axis of handle 202 of medical device 200 and in a direction parallel to an axis of rotation of any of the gears 831, 845, 841) together as a single unit relative to gears 860, 862 and first spool assembly 220, but may rotate independently of one another. Gear unit 829 may be coupled to an actuator (e.g., a lever or other actuator) configured to move gear unit 829 laterally.

Lateral movement of gear unit 829 relative to gears 860, 862 and first spool assembly 220 may change which gear of gear unit 829 engages (e.g., meshes) with gears 862 and 288, and may thereby change a gear ratio, e.g., between second gear 862 and gear unit 829 or between gear unit 829 and gear 288. As more clearly shown in FIGS. 8B-8D, compound gears 831, 845 and single gear 841 may be arranged proximally or distally offset from one another, which may help facilitate alignment of first compound gear 831, second compound gear 845, or single gear 841 with gears 862 and 288, e.g., when gear unit 829 is moved laterally to change gear ratios. For example, as shown in FIG. 8B, rotation axes G, H, and I of first compound gear 831, single gear 841, and second compound gear 845, respectively, may be proximally or distally offset from one another.

FIGS. 8A-8D illustrate gear unit 829 in three different positions that provide three different gear ratios. FIGS. 8A and 8B illustrate gear unit 829 in a first position that provides a 1:1 gear ratio (e.g., a neutral gear ratio). FIG. 8C illustrates gear unit 829 in a second position that provides a high gear ratio. FIG. 8D illustrates gear unit 829 in a third position that provides a low gear ratio.

As shown in FIGS. 8A and 8B, in the first position, single gear 841 is disposed between and aligned with gears 862 and 288 such that external teeth 843 of single gear 841 mesh with external teeth 868 of first gear 860 and with the external teeth of gear 288. In some aspects, a diameter of single gear 841 may be the same as or approximately the same as a diameter of gear 288, to provide a neutral gear ratio. Single gear 841 may have a same thickness in a lateral direction as compound gears 831, 845 to facilitate transition among the three positions.

As shown in FIG. 8C, in the second position, first compound gear 831 may be disposed between and aligned with gears 862 and 288 such that external teeth 835 of large gear 833 mesh with the external teeth of gear 288, while external teeth 839 of small gear 837 mesh with external teeth 870 of second gear 862. A diameter of small gear 837 may be smaller than a diameter of gear 288, and gear 841. Thus, first compound gear 831 may rotate faster/a greater angular amount as compared to single gear 841 for a same given amount of rotation of second control knob 224 (and first gear 860 and second gear 862). This relatively greater rotation of first compound gear 831 for a given rotation of knob 224 may result in greater rotation of gear 288 and spool 278 via large gear 833, and thus greater articulation or deflection of steerable section 216 (and thus distal tip 208) of shaft 206. For example, as compared with the first position, a same amount of rotation of control knob 224 may produce greater articulation of the steerable section 216 (and thus distal tip 208) of shaft 206. In the second position, an operator may be required to apply more force on control knob 224 as compared to when gear unit 829 is in the first position. This second position may be akin to a higher gear on a bicycle and may be similar to the configuration of FIG. 3B.

As shown in FIG. 8D, in the third position, second compound gear 845 may be disposed between and aligned with gears 862 and 288 such that external teeth 849 of large gear 847 mesh with external teeth 870 of second gear 862, while external teeth 853 of small gear 851 mesh with the external teeth of gear 288. A diameter of large gear 847 may be larger than a diameter of single gear 841, small gear 837, and gear 288. Thus, second compound gear 845 may rotate slower/a smaller angular amount as compared to single gear 841 for a same given amount of rotation of second control knob 224 (and first gear 860 and second gear 862). In some aspects, small gears 837 and 851 may have approximately the same diameter, and large gears 833 and 847 may have approximately the same diameter. This relatively smaller rotation of second compound gear 845 for a given rotation of knob 224 may result in smaller rotation of gear 288 and spool 278 via small gear 851, and thus less articulation or deflection of steerable section 216 of shaft 206. For example, as compared with the first position and the second position, a same amount of rotation of control knob 224 may produce less articulation of the steerable section 216 (and thus distal tip 208) of shaft 206. In the third position, an operator may be required to apply less force on control knob 224 as compared to when gear unit 829 is in the first position. This second position may be akin to a lower gear on a bicycle and may be similar to the configuration of FIG. 5B.

An exemplary method of using medical device 200 with gear assembly 895 in place of first gear assembly 250 will now be described. With gear unit 829 in the first position illustrated in FIGS. 8A and 8B, an operator may rotate second control knob 224 clockwise about rotation axis A, which may cause first gear 860 to also rotate clockwise about rotation axis A. Clockwise rotation of first gear 860 about rotation axis A may cause external teeth 868 of first gear 860 to mesh with external teeth 870 of second gear 862 and rotate second gear 862 counterclockwise about a rotation axis F. Counterclockwise rotation of second gear 862 about rotation axis F may cause external teeth 870 of second gear 862 to mesh with external teeth 843 of single gear 841 and rotate single gear 841 clockwise about rotation axis H. Clockwise rotation of single gear 841 about rotation axis H may cause external teeth 843 of single gear 841 to engage with the external teeth of gear 288 and rotate first spool assembly 220 (gear 288 and spool 278) counterclockwise about rotation axis D. Counterclockwise rotation of first spool assembly 220 about rotation axis D may move first and second pull wires 292, 294 to deflect steerable section 216 (and thus distal tip 208) of shaft 206 in the up or down direction.

Optionally, the operator may change gear ratios by actuating the actuator coupled to gear unit 829. For example, the operator may actuate the actuator to move gear unit 829 laterally from the first position (FIGS. 8A and 8B) to the second position (FIG. 8C). With gear unit 829 now in the second position illustrated in FIG. 8C, the operator may rotate second control knob 224 clockwise about rotation axis A, which may cause first gear 860 to also rotate clockwise about rotation axis A. Clockwise rotation of first gear 860 about rotation axis A may cause external teeth 868 of first gear 860 to engage with external teeth 870 of second gear 862 and rotate second gear 862 counterclockwise about rotation axis F. Counterclockwise rotation of second gear 862 about rotation axis F may cause external teeth 870 of second gear 862 to engage with external teeth 839 of small gear 837 and rotate first compound gear 831 clockwise about rotation axis G. Clockwise rotation of first compound gear 831 about rotation axis G may cause external teeth 835 of large gear 833 to mesh with the external teeth of gear 288 and rotate first spool assembly 220 (gear 288 and spool 278) counterclockwise about rotation axis D. Counterclockwise rotation of first spool assembly 220 about rotation axis D may move first and second pull wires 292, 294 to deflect steerable section 216 of shaft 206 in the up or down direction. Although one direction of movement is described above, opposite directions of movement of knob 224 may result in opposite direction of movement of other components.

It will be appreciated that the operator may change gear ratios multiple times throughout a medical procedure. For example, gear unit 829 may be moved laterally from the first position (FIGS. 8A and 8B) to the second position (FIG. 8C), from the first position to the third position (FIG. 8D), from the second position to the first position, from the third position to the first position, from the second position to the third position, or from the third position to the second position. The operator may change the gear ratio based on the operator’s preferences or as necessitated by the medical procedure (e.g., more or less sensitive articulation of distal tip 208 or more or less force required to be exerted on control knob 224 as discussed above).

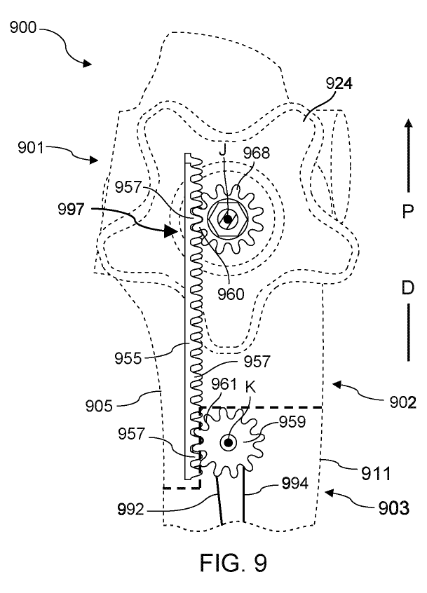

FIG. 9 illustrates features of an exemplary medical device 900 that includes a reusable portion 901 removably coupled to a disposable portion 903. In particular, FIG. 9 illustrates internal components of a handle 902 that is formed when a housing 905 of reusable portion 901 is coupled to a housing 911 of disposable portion 903. Reusable portion 901 may include any of the features of reusable portions 201, 301, 401, 501, unless otherwise specified herein, and disposable portion 903 may include any of the features of disposable portion 203, unless otherwise specified herein. For example, disposable portion 903 may include a shaft (similar to shaft 206) extending distally from housing 911 to a deflectable distal tip (similar to distal tip 208).

Reusable portion 901 may include control knobs including a second control knob 924 for deflecting the distal tip of disposable portion 903 in an up or down direction. Reusable portion 901 may also include a first control knob (not shown for ease of illustration) for deflecting the distal tip of disposable portion 903 in a left or right direction. Reusable portion 901 may include a gear assembly 997 within housing 905. Gear assembly 997 may include a first gear 960 and a rack gear 955. Gear assembly 997 may also include other gears (e.g., idler gears, such as those described in other embodiments herein. First gear 960 may be rotatably fixedly coupled to second control knob 924. Rack gear 955 may extend substantially parallel to a central longitudinal axis of handle 902, and teeth 957 of rack gear 955 may be engaged with external teeth 968 of first gear 960. For example, rack gear 955 may extend distally from a proximal end engaged with first gear 960 to a distal end proximate a distal end of housing 905. Rack gear 955 may be longitudinally movable, e.g., movable in a proximal or distal direction.

Disposable portion 903 may include a gear 959 within housing 911. Gear 959 may be coupled to first and second pull wires 992, 994 for deflecting the distal tip of disposable portion 903. For example, gear 959 may be coupled to a spool having properties of any of the spools disclosed herein. For example, gear 959 may be coupled to a spool forming a spool assembly having properties of any of the spool assemblies disclosed herein. Gear 959 may be configured to engage (e.g., removably engage) with rack gear 955. As shown in FIG. 9, when housing 905 of reusable portion 901 is connected to housing 911 of disposable portion 903, teeth 957 of rack gear 955 may be engaged with external teeth 961 of gear 959. For example, gear 959 may be engaged with the distal end of rack gear 955.

In use, an operator may rotate second control knob 924 counterclockwise about a rotation axis J, which may cause first gear 960 to rotate counterclockwise about rotation axis J. Counterclockwise rotation of first gear 960 about rotation axis J may cause external teeth 968 of first gear 960 to mesh with teeth 957 of rack gear 955, which in turn may cause rack gear 955 to move distally. Distal movement of rack gear 955 may cause teeth 957 of rack gear 955 to mesh with external teeth 961 of gear 959, which in turn may cause gear 959 to rotate counterclockwise about a rotation axis K. Counterclockwise rotation of gear 959 about rotation axis K may cause first and second pull wires 992, 994 to move (e.g., move proximally or distally) to deflect the distal tip of disposable portion 903 in the up or down direction. Although one direction of movement is described above, opposite directions of movement of knob 924 may result in opposite direction of movement of other components.

It will be appreciated that reusable portion 901 may include a second gear assembly 997 coupled to the first control knob, and disposable portion 903 may include a second gear 959 and a second set of pull wires 992, 994. The second gear 959 may be removably engaged with the second gear assembly 997 for deflecting the distal tip of disposable portion 903 in the left or right direction.

FIG. 10 illustrates a schematic, partially cross-sectional view of features of an exemplary medical device 1000 that includes a reusable portion 1002 and a disposable portion 1006. Reusable portion 1002 may include a housing 1004 configured to be removably coupled to a housing 1008 of disposable portion 1006 to form a handle of medical device 1000. Reusable portion 1002 may include any of the features of reusable portions 201, 301, 401, 501, 901, unless otherwise specified herein, and disposable portion 1006 may include any of the features of disposable portions 203, 903, unless otherwise specified herein. For example, disposable portion 1006 may include a shaft having any of the features of shaft 206. The shaft of disposable portion 1006 may extend distally from housing 1008 to a deflectable distal tip (similar to distal tip 208).

Reusable portion 1002 may include a first control knob 1022 for deflecting the distal tip of disposable portion 1006 in a left or right direction, and may include a second control knob 1024 for deflecting the distal tip of disposable portion 1006 in an up or down direction. Reusable portion 1002 may include a first axle or shaft 1010 (e.g., a mounting shaft) that is rotatably or fixedly coupled to structure within housing 1004, a second shaft 1012 that surrounds first shaft 1010, and a third shaft 1014 that surrounds second shaft 1012. Second shaft 1012 may be coupled to first control knob 1022 (e.g., rotationally fixed to first control knob 1022), and third shaft 1014 may be coupled to second control knob 1024 (e.g., rotationally fixed to second control knob 1024). Shafts 1010, 1012, and 1014 may be concentric. Second and third shafts 1012, 1014 may rotate about a rotation axis M independently of one another (with rotation of first control knob 1022 and second control knob 1024, respectively) and relative to shaft first 1010. For example, rotation of first control knob 1022 about rotation axis M may rotate second shaft 1012 about rotation axis M, but may not rotate third shaft 1014 (and second control knob 1024) about rotation axis M. Similarly, rotation of second control knob 1024 about rotation axis M may rotate third shaft 1014 about rotation axis M, but may not rotate second shaft 1012 (and first control knob 1022) about rotation axis M.

Second shaft 1012 may be fixedly coupled (e.g., rotationally fixedly coupled) to a first gear 1016 (e.g., a bevel gear), and third shaft 1014 may be fixedly coupled (e.g., rotationally fixedly coupled) to a second gear 1018 (e.g., a bevel gear). For example, first gear 1016 may rotate with second shaft 1012 and first control knob 1022 about rotation axis M, and second gear 1018 may rotate with third shaft 1014 and second control knob 1024 about rotation axis M. First gear 1016 may surround first shaft 1010, and second gear 1018 may surround second shaft 1012. For example, first and second gears 1016, 1018 may be coaxial.

Reusable portion 1002 may further include a fourth shaft 1020 and a fifth shaft 1026 within housing 1004. Fourth and fifth shafts 1020, 1026 may each extend substantially parallel to a longitudinal axis of housing 1004, and substantially parallel to one another. Fourth and fifth shafts 1020, 1026 may each have a non-round/non-circular cross-sectional shape. For example, fourth and fifth shafts 1020, 1026 may each have an X-cross-sectional shape as illustrated in FIG. 10; however, such a cross-sectional shape is merely exemplary and any suitable non-round/non-circular cross-sectional shape may be utilized (e.g., oval, elongate, polygonal, star, etc.). In some aspects, proximal portions of fourth and fifth shafts 1020, 1026 may have a round cross-sectional shape, and distal portions of fourth and fifth shafts 1020, 1026 may have a non-round cross-sectional shape. In some examples, fourth and fifth shafts 1020, 1026 may be spline shafts.

Reusable portion 1002 may further include a third gear 1028 (e.g., a bevel gear) fixedly coupled (e.g., rotationally fixedly coupled) to fourth shaft 1020, and a fourth gear 1030 (e.g., a bevel gear) fixedly coupled (e.g., rotationally fixedly coupled) to fifth shaft 1026. For example, third gear 1028 may be fixedly coupled to a proximal end of fourth shaft 1020, and fourth gear 1030 may be fixedly coupled to a proximal end of fifth shaft 1026.

Third gear 1028 and fourth shaft 1020 may be gearingly coupled to first gear 1016 and second shaft 1012. Third gear 1028 may be positioned in contact with and substantially perpendicular to first gear 1016, such that first gear 1016 may mesh with third gear 1028 to transfer torque to fourth shaft 1020 at an approximately 90-degree angle. In use, rotation of first control knob 1022 about rotation axis M in a direction (e.g., clockwise or counterclockwise) may cause second shaft 1012/first gear 1016 to also rotate about rotation axis M in the same direction. Rotation of second shaft 1012/first gear 1016 about rotation axis M may cause first gear 1016 to mesh with third gear 1028, and thereby rotate third gear 1028/fourth shaft 1020 about a rotation axis N. Rotation axis N may be substantially perpendicular to rotation axis M and may be substantially parallel to a central longitudinal axis of reusable portion 1002.

Fourth gear 1030 and fifth shaft 1026 may be gearingly coupled to second gear 1018 and third shaft 1014. Fourth gear 1030 may be positioned in contact with and substantially perpendicular to second gear 1018, such that second gear 1018 may mesh with fourth gear 1030 to transfer torque to fifth shaft 1026 at an approximately 90-degree angle. In use, rotation of second control knob 1024 about rotation axis M in a direction (e.g., clockwise or counterclockwise) may cause third shaft 1014/second gear 1018 to also rotate about rotation axis M in a same direction. Rotation of third shaft 1014/second gear 1018 about rotation axis M may cause second gear 1018 to mesh with fourth gear 1030, and thereby rotate fourth gear 1030/fifth shaft 1026 about rotation axis P. Rotation axis P may be substantially perpendicular to rotation axis M and substantially parallel to rotation axis N and the central longitudinal axis of reusable portion 1002.

Disposable portion 1006 may include a first shaft 1032 and a second shaft 1034 within housing 1008. First and second shafts 1032, 1034 may each extend substantially parallel to a central longitudinal axis of disposable portion 1006, and substantially parallel to one another. For example, first shaft 1032 may extend along axis N, and second shaft 1034 may extend along axis P. First shaft 1032 may be fixedly coupled (e.g., rotationally fixedly coupled) to a first gear 1036 (e.g., a bevel gear), and second shaft 1034 may be fixedly coupled (e.g., rotationally fixedly coupled) to a second gear 1038 (e.g., a bevel gear). For example, first gear 1036 may be fixedly coupled to a distal end of first shaft 1032, and second gear 1038 may be fixedly coupled to a distal end of second shaft 1034.

First and second shafts 1032, 1034 of disposable portion 1006 may be configured to receive or otherwise mate with fourth and fifth shafts 1020, 1026 of reusable portion 1002, respectively. First shaft 1032 may define an opening, recess, or lumen 1048 that is configured to receive fourth shaft 1020, and second shaft 1034 may define an opening, recess, or lumen 1050 that is configured to receive fifth shaft 1026. A proximal end of first shaft 1032 may include an opening in communication with lumen 1048 to facilitate insertion of fourth shaft 1020 into lumen 1048. Similarly, a proximal end of second shaft 1034 may include an opening in communication with lumen 1050 to facilitate insertion of fifth shaft 1026 into lumen 1050.

A cross-sectional shape of lumen 1048 of first shaft 1032 may be complementary to a cross-sectional shape of fourth shaft 1020, such that fourth shaft 1020 may be keyed into lumen 1048. Consequently, rotation of third gear 1028/fourth shaft 1020 about rotation axis N may cause rotation of first shaft 1032/first gear 1036 about rotation axis N when fourth shaft 1020 is received within lumen 1048 of first shaft 1032. A cross-sectional shape of lumen 1050 of second shaft 1034 may be complementary to a cross-sectional shape of fifth shaft 1026, such that fifth shaft 1026 may be keyed into lumen 1050. Consequently, rotation of fourth gear 1030/fifth shaft 1026 about rotation axis P may cause rotation of second shaft 1034/second gear 1038 about rotation axis P when fifth shaft 1026 is received within lumen 1050 of second shaft 1034. Lumens 1048, 1050 may each have a X-cross-sectional shape as illustrated in FIG. 10; however, such a cross-sectional shape is merely exemplary and any suitable non-round/non-circular cross-sectional shape may be utilized (e.g., oval, elongate, polygonal, star, etc.).

In aspects, fourth shaft 1020 may include an opening, recess, or lumen, and first shaft 1032 may be inserted into fourth shaft 1020. Similarly, fifth shaft 1026 may include an opening, recess, or lumen, and second shaft 1034 may be inserted into fifth shaft 1026. In other aspects, the shafts may be mated by other mechanisms.

Disposable portion 1006 may further include a third gear 1040, a fourth gear 1042, a first spool 1017, a second spool 1019, a first set of two pull wires 1044, and a second set of two pull wires 1046.

First shaft 1032/first gear 1036 may be coupled to third gear 1040, first spool 1017, and pull wires 1044. Third gear 1040 may be positioned in contact with and substantially perpendicular to first gear 1036, such that first shaft 1032/first gear 1036 may transfer torque to an axle/shaft of third gear 1040 at an approximately 90-degree angle. Pull wires 1044 may be coupled to and extend distally from first spool 1017, through the shaft of disposable portion 1006, and to the distal tip of disposable portion 1006. Third gear 1040 may be fixedly coupled (e.g., rotationally fixedly coupled) to first spool 1017, such that rotation of third gear 1040 about a rotation axis S in a direction (e.g., clockwise or counterclockwise) also rotates first spool 1017 about rotation axis S in a same direction. Rotation of third gear 1040/first spool 1017 (e.g., a spool assembly) about rotation axis S may cause pull wires 1044 to move (e.g., move pull wires 1044 proximally or distally) and thereby deflect the distal tip of disposable portion 1006 in the left or right direction. Rotation axis S may be substantially perpendicular to rotation axis N and axis P and may be substantially parallel to axis M.

Second shaft 1034/second gear 1038 may be coupled to fourth gear 1042, second spool 1019, and pull wires 1046. Fourth gear 1040 may be positioned in contact with and substantially perpendicular to second gear 1038, such that second shaft 1034/second gear 1038 may transfer torque to an axle/shaft of fourth gear 1042 at an approximately 90-degree angle. Pull wires 1046 may be coupled to and extend distally from second spool 1019, through the shaft of disposable portion 1006, and to the distal tip of disposable portion 1006. Fourth gear 1042 may be fixedly coupled (e.g., rotationally fixedly coupled) to second spool 1019, such that rotation of fourth gear 1042 about rotation axis S in a direction (e.g., clockwise or counterclockwise) also rotates second spool 1019 about rotation axis S in the same direction. Rotation of fourth gear 1042/second spool 1019 (e.g., a spool assembly) about rotation axis S may cause second pull wires 1046 to move (e.g., move pull wires 1046 proximally or distally) and thereby cause deflection of the distal tip of disposable portion 1006 in the up or down direction.

As shown in FIG. 10, third gear 1040 and fourth gear 1042 may be disposed within and on opposite sides of housing 1008. First spool 1017 may be disposed on a side of third gear 1040 closest to a center of housing 1008. In other examples, first spool 1017 may be disposed on a side of third gear 1040 furthest from the center of housing 1008. Similarly, as shown in FIG. 10, second spool 1019 may be disposed on a side of fourth gear 1042 closest to a center of housing 1008. In other examples, second spool 1019 may be disposed on a side of fourth gear 1042 furthest from the center of housing 1008. Although third gear 1040/first spool 1017 and fourth gear 1042/second spool 1019 are described as having the same rotation axis S, in other examples, third gear 1040/first spool 1017 and fourth gear 1042/second spool 1019 may not be coaxial.

In use, an operator may rotate first control knob 1022 about rotation axis M in a direction (e.g., clockwise or counterclockwise), which may rotate second shaft 1012/first gear 1016 about rotation axis M in a same direction. Rotation of second shaft 1012/first gear 1016 about rotation axis M may cause third gear 1028/fourth shaft 1020 to rotate about rotation axis N in a direction (e.g., clockwise or counterclockwise), which in turn may cause first shaft 1032/first gear 1036 to also rotate about rotation axis N in the same direction. Rotation of first shaft 1032/first gear 1036 about rotation axis N may cause third gear 1040/first spool 1017 to rotate about rotation axis S to move pull wires 1044 (e.g., move pull wires 1044 proximally or distally). Movement of pull wires 1044 may deflect the distal tip of disposable portion 1006 in the left or right direction.

In use, an operator may rotate second control knob 1024 about rotation axis M in a direction (e.g., clockwise or counterclockwise), which may rotate third shaft 1014/second gear 1018 about rotation axis M in the same direction. Rotation of third shaft 1014/second gear 1018 about rotation axis M may cause fourth gear 1030/fifth shaft 1026 to rotate about rotation axis P in a direction (e.g., clockwise or counterclockwise), which in turn may cause second shaft 1034/second gear 1038 to also rotate about rotation axis P in the same direction. Rotation of second shaft 1034/second gear 1038 about rotation axis P may cause fourth gear 1042/second spool 1019 to rotate about rotation axis S and move pull wires 1046 (e.g., move pull wires 1046 proximally or distally). Movement of pull wires 1046 may deflect the distal tip of disposable portion 1006 in the up or down direction. In alternatives, first control knob 1022 may control up/down movement, and second control knob 1024 may control left/right movement. Rotation of first and second control knobs 1022, 1024 in an opposite direction to that described above may result in opposite directions of rotation of the other elements described above.

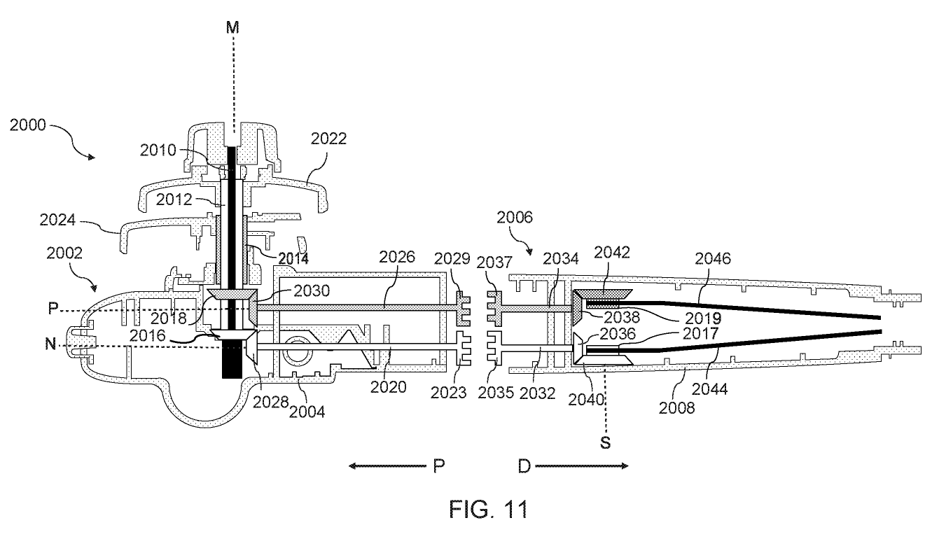

FIG. 11 illustrates a schematic, partially cross-sectional view of features of an exemplary medical device 2000 that includes a reusable portion 2002 and a disposable portion 2006. Reusable portion 2002 may include a housing 2004 configured to be removably coupled to a housing 2008 of disposable portion 2006 to form a handle of medical device 2000. Reusable portion 2002 may include any of the features of reusable portions 201, 301, 401, 501, 901, 1002, unless otherwise specified herein, and disposable portion 2006 may include any of the features of disposable portions 203, 903, 1006, unless otherwise specified herein. For example, disposable portion 2006 may include a shaft having any of the features of shaft 206. The shaft of disposable portion 2006 may extend distally from housing 2008 to a deflectable distal tip (similar to distal tip 208).

Reusable portion 2002 may include a first control knob 2022 similar to first control knob 1022 for deflecting the distal tip of disposable portion 2006 in a left or right direction, and may include a second control knob 2024 similar to second control knob 2024 for deflecting the distal tip of disposable portion 2006 in an up or down direction. Reusable portion 2002 may include a first axle or shaft 2010 (e.g., a mounting shaft) similar to first shaft 1010, a second shaft 2012 (similar to second shaft 1012) coupled to first control knob 2022, and a third shaft 2014 (similar to third shaft 1014) coupled to second control knob 2024. Second shaft 2012 may be fixedly coupled (e.g., rotationally fixedly coupled) to a first gear 2016 (e.g., a bevel gear) similar to first gear 1016, and third shaft 2014 may be fixedly coupled (e.g., rotationally fixedly coupled) to a second gear 2018 (e.g., a bevel gear) similar to second gear 1018.

Reusable portion 2002 may further include a fourth shaft 2020 and a fifth shaft 2026 within housing 2004. Fourth and fifth shafts 2020, 2026 may each extend substantially parallel to a longitudinal axis of housing 2004, and may extend substantially parallel to one another. A proximal end of fourth shaft 2020 may be fixedly coupled (e.g., rotationally fixedly coupled) to a third gear 2028 (e.g., a bevel gear) similar to third gear 1028. A proximal end of fifth shaft 2026 may be fixedly coupled (e.g., rotationally fixedly coupled) to a fourth gear 2030 similar to fourth gear 1030.

In this example, fourth and fifth shafts 2020, 2026 may each have any suitable cross-sectional shape (e.g., round/circular, non-round/non-circular, oval, elongate, polygonal, star, etc.). In this example, a distal end of fourth shaft 2020 may be fixedly coupled (e.g., rotationally fixedly coupled) to a first coupler 2023, and a distal end of fifth shaft 2026 may be fixedly coupled (e.g., rotationally fixedly coupled) to a second coupler 2029.

Third gear 2028/fourth shaft 2020/first coupler 2023 may be gearingly coupled to first gear 2016/second shaft 2012. Third gear 2028 may be positioned in contact with and substantially perpendicular to first gear 2016, such that first gear 2016 may mesh with third gear 2028 to transfer torque to fourth shaft 2020 at an approximately 90-degree angle. In use, rotation of first control knob 2022 about rotation axis M in a direction (e.g., clockwise or counterclockwise) may cause second shaft 2012/first gear 2016 to also rotate about rotation axis M in the same direction. Rotation of second shaft 2012/first gear 2016 about rotation axis M may cause first gear 2016 to mesh with third gear 2028, and thereby rotate third gear 2028/ fourth shaft 2020/first coupler 2023 about rotation axis N.

Fourth gear 2030/fifth shaft 2026/second coupler 2029 may be gearingly coupled to second gear 2018/third shaft 2014. Fourth gear 2030 may be positioned in contact with and substantially perpendicular to second gear 2018, such that second gear 2018 may mesh with fourth gear 2030 to transfer torque to fifth shaft 2026 at an approximately 90-degree angle. In use, rotation of second control knob 2024 about rotation axis M in a direction (e.g., clockwise or counterclockwise) may cause third shaft 2014/second gear 2018 to also rotate about rotation axis M in the same direction. Rotation of third shaft 2014/second gear 2018 about rotation axis M may cause second gear 2018 to mesh with fourth gear 2030, and thereby rotate fourth gear 2030/fifth shaft 2026/second coupler 2029 about rotation axis P.

Disposable portion 2006 may include a first shaft 2032 and a second shaft 2034 within housing 2008. First and second shafts 2032, 2034 may each extend substantially parallel to a longitudinal axis of disposable portion 2006, and may extend substantially parallel to one another. A distal end of first shaft 2032 may be fixedly coupled (e.g., rotationally fixedly coupled) to a first gear 2036 (e.g., a bevel gear) similar to first gear 1036. A distal end of second shaft 2034 may be fixedly coupled (e.g., rotationally fixedly coupled) to a second gear 2038 similar to second gear 1038.

In this example, a proximal end of first shaft 2032 may be fixedly coupled (e.g., rotationally fixedly coupled) to a first coupler 2035, and a proximal end of second shaft 2034 may be fixedly coupled (e.g., rotationally fixedly coupled) to a second coupler 2037. First and second couplers 2023, 2029 of reusable portion 2002 may be configured to removably engage with or connect to first and second couplers 2035, 2037 of disposable portion 2006, respectively. For example, a shape of first coupler 2023 of reusable portion 2002 may be complementary to a shape of first coupler 2035 of disposable portion 2006, and a shape of second coupler 2029 of reusable portion 2002 may be complementary to a shape of second coupler 2037 of disposable portion 2006.

First coupler 2023 of reusable portion 2002 may be removably engageable with or connectable to first coupler 2035 of disposable portion 2006 to transfer torque to first shaft 2032. For example, rotation of third gear 2028/fourth shaft 2020/first coupler 2023 about rotation axis N in a direction (e.g., clockwise or counterclockwise) may cause rotation of first coupler 2035/first shaft 2032/first gear 2036 about rotation axis N in the same direction. In other examples, a rotation axis of first coupler 2035/first shaft 2032/first gear 2036 may be offset or different from a rotation axis of third gear 2028/fourth shaft 2020/first coupler 2023.

Second coupler 2029 of reusable portion 2002 may be removably engageable with or connectable to second coupler 2037 of disposable portion 2006 to transfer torque to second shaft 2034. For example, rotation of fourth gear 2030/fifth shaft 2026/second coupler 2029 about rotation axis P in a direction (e.g., clockwise or counterclockwise) may cause rotation of second coupler 2037/second shaft 2034/second gear 2038 about rotation axis P in the same direction. In other examples, a rotation axis of second coupler 2037/second shaft 2034/second gear 2038 may be offset or different from a rotation axis of fourth gear 2030/fifth shaft 2026/second coupler 2029.

In some examples, the coupling or connection between couplers 2023 and 2035 or between couplers 2029 and 2037 may resemble or may be a face gear coupling, a parallel jaw coupling, a universal coupling, an Oldham coupling, or a friction plate coupling. In aspects, first coupler 2023 and second coupler 2035 may have interlocking teeth or any of the shapes described above with respect to medical device 1000. Similarly, second coupler 2029 and second coupler 2037 may have interlocking teeth or any of the shapes described above with respect to medical device 1000.

Disposable portion 2006 may further include a third gear 2040 (e.g., a bevel gear) similar to third gear 1040, a fourth gear 2038 (e.g., a bevel gear) similar to fourth gear 1038, a first spool 2017 similar to first spool 1017, a second spool 2019 similar to second spool 1019, a first set of two pull wires 2044 similar to first set of two pull wires 1044, and a second set of two pull wires 2046 similar to second set of two pull wires 1046. For example, third gear 2040 may be positioned in contact with and substantially perpendicular to first gear 2036, and may be fixedly coupled (e.g., rotationally fixedly coupled) to first spool 2017. Pull wires 2044 may be coupled to and extend distally from first spool 2017, through the shaft of disposable portion 2006, and to the distal tip of disposable portion 2006. For example, fourth gear 2042 may be positioned in contact with and substantially perpendicular to second gear 2038, and may be fixedly coupled (e.g., rotationally fixedly coupled) to second spool 2019. Pull wires 2046 may be coupled to and extend distally from second spool 2019, through the shaft of disposable portion 2006, and to the distal tip of disposable portion 2006.

In use, an operator may rotate first control knob 2022 about rotation axis M in a direction (e.g., clockwise or counterclockwise), which may rotate second shaft 2012/first gear 2016 about rotation axis M in the same direction. Rotation of second shaft 2012/first gear 2016 about rotation axis M may cause third gear 2028/fourth shaft 2020/first coupler 2023 to rotate about rotation axis N in a direction (e.g., clockwise or counterclockwise), which in turn may cause first coupler 2035/first shaft 2032/first gear 2036 to also rotate about rotation axis N in the same direction. Rotation of first coupler 2035/first shaft 2032/first gear 2036 about rotation axis N may cause third gear 2040/first spool 2017 (e.g., a spool assembly) to rotate about rotation axis S and move pull wires 2044 (e.g., move pull wires 2044 proximally or distally). Movement of pull wires 2044 may deflect the distal tip of disposable portion 2006 in the left or right direction.

In use, an operator may rotate second control knob 2024 about rotation axis M in a direction (e.g., clockwise or counterclockwise), which may rotate third shaft 2014/second gear 2018 about rotation axis M in the same direction. Rotation of third shaft 2014/second gear 2018 about rotation axis M may cause fourth gear 2030/fifth shaft 2026/second coupler 2029 to rotate about rotation axis P in a direction (e.g., clockwise or counterclockwise), which in turn may cause second coupler 2037/second shaft 2034/second gear 2038 to also rotate about rotation axis P in the same direction. Rotation of second coupler 2037/second shaft 2034/second gear 2038 about rotation axis P may cause fourth gear 2042/second spool 2019 (e.g., a spool assembly) to rotate about rotation axis S and move pull wires 2046 (e.g., move pull wires 2046 proximally or distally). Movement of pull wires 2046 may deflect the distal tip of disposable portion 2006 in the up or down direction.

In alternatives, first control knob 2022 may control up/down movement, and second control knob 2024 may control left/right movement. Rotation of first and second control knobs 2022, 2024 in an opposite direction to that described above may result in opposite directions of rotation of the other elements described above.

As shown in FIG. 11, third gear 2040 and fourth gear 2042 may be disposed within and on opposite sides of housing 2008. First spool 2017 may be disposed on a side of third gear 2040 closest to a center of housing 2008. In other examples, first spool 2017 may be disposed on a side of third gear 2040 furthest from the center of housing 2008. Similarly, as shown in FIG. 11, second spool 2019 may be disposed on a side of fourth gear 2042 closest to a center of housing 2008. In other examples, second spool 2019 may be disposed on a side of fourth gear 2042 furthest from the center of housing 2008. Although third gear 2040/first spool 2017 and fourth gear 2042/second spool 2019 are described as rotating about the same rotation axis S, in other examples, third gear 2040/first spool 2017 and fourth gear 2042/second spool 2019 may not be coaxial.

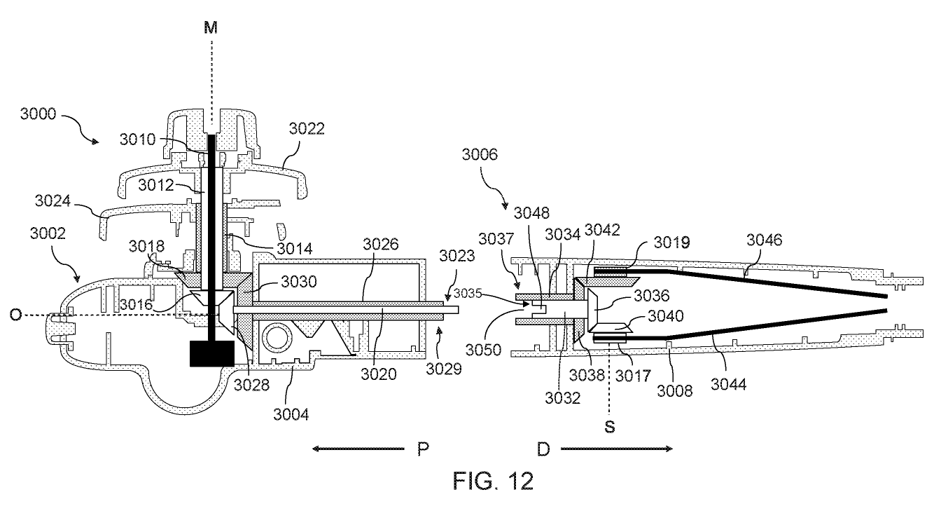

FIG. 12 illustrates a schematic, partially cross-sectional view of features of an exemplary medical device 3000 that includes a reusable portion 3002 and a disposable portion 3006. Reusable portion 3002 may include a housing 3004 configured to be removably coupled to a housing 3008 of disposable portion 3006 to form a handle of medical device 3000. Reusable portion 3002 may include any of the features of reusable portions 201, 301, 401, 501, 901, 1002, 2002, unless otherwise specified herein, and disposable portion 3006 may include any of the features of disposable portions 203, 903, 1006, 2006, unless otherwise specified herein. For example, disposable portion 3006 may include a shaft having any of the features of shaft 206. The shaft of disposable portion 3006 may extend distally from housing 3008 to a deflectable distal tip (similar to distal tip 208).

Reusable portion 3002 may include a first control knob 3022 similar to first control knobs 1022, 2022 for deflecting the distal tip of disposable portion 3006 in a left or right direction, and may include a second control knob 3024 similar to second control knobs 1024, 2024 for deflecting the distal tip of disposable portion 3006 in an up or down direction. Reusable portion 3002 may include a first shaft 3010 (e.g., a mounting shaft) similar to first shafts 1010, 2010, a second shaft 3012 (similar to second shafts 1012, 2012) coupled to first control knob 3022, and a third shaft 3014 (similar to third shafts 1014, 2014) coupled to second control knob 3024. Second shaft 3012 may be fixedly coupled (e.g., rotationally fixedly coupled) to a first gear 3016 (e.g., a bevel gear), and third shaft 3014 may be fixedly coupled (e.g., rotationally fixedly coupled) to a second gear 3018 (e.g., a bevel gear). First gear 3016 may surround first shaft 3010, and second gear 3018 may surround second shaft 2012. First gear 3016 may be radially inward relative to second gear 3018. First gear 3016 may be smaller (e.g., have a smaller diameter) than second gear 3018.

Reusable portion 3002 may further include a fourth shaft 3020 and a fifth shaft 3026 that may rotate independently of one another. Fourth and fifth shafts 3020, 3026 may extend substantially parallel to a longitudinal axis of housing 3004. In this example, fourth shaft 3020 may extend through fifth shaft 3026, and through a fourth gear 3030. For example, fourth and fifth shafts 3020, 3026 may be concentric.

A proximal end fourth shaft 3020 may be fixedly coupled (e.g., rotationally fixedly coupled) to a third gear 3028, and a proximal end of fifth shaft 3026 may be fixedly coupled (e.g., rotationally fixedly coupled) to fourth gear 3030 (e.g., a bevel gear). Third gear 3028 may be proximal to fourth gear 3030. Third gear 3028 may be smaller than (e.g., have a smaller diameter) than fourth gear 3030. Third gear 3028/fourth shaft 3020 may be coupled to first gear 3016/second shaft 3012, and fourth gear 3030/fifth shaft 3026 may be coupled to second gear 3018/third shaft 3014. For example, third gear 3028 may be positioned in contact with and substantially perpendicular to first gear 3016, and fourth gear 3030 may be positioned in contact with and substantially perpendicular to second gear 3018.

In this example, first and third gears 3016, 3028 may be nested within and may rotate independently of second and fourth gears 3018, 3030. For example, first and second gears 3016, 3018 may be coaxial, and third and fourth gears 3028, 3030 may be coaxial. As shown in FIG. 12, a distal portion 3023 of fourth shaft 3020 may be distal to a distal end 3029 of fifth shaft 3026. Similar to shaft 1026, a cross-sectional shape of fifth shaft 3026 may be any suitable non-round/non-circular cross-sectional shape, and a cross-sectional shape of distal portion 3023 of fourth shaft 3020 may be any suitable non-round/non-circular cross-sectional shape. For example, distal portion 3023 of fourth shaft 3020 may have a non-round/non-circular cross-sectional shape, while remaining portions of fourth shaft 3020 may have a round/circular cross-sectional shape. Alternatively, an entirety of fourth shaft 3020 may have a non-round/non-circular cross-sectional shape, while permitting fourth and fifth shafts 3020, 3026 to rotate independently of one another. In some examples, fourth and fifth shafts 3020, 3026 may be spline shafts.

In use, rotation of first control knob 3022 about rotation axis M in a direction (e.g., clockwise or counterclockwise) may cause second shaft 3012/first gear 3016 to also rotate about rotation axis M in the same direction. Rotation of second shaft 3012/first gear 3016 about rotation axis M may cause first gear 3016 to mesh with third gear 3028, and thereby rotate third gear 3028/fourth shaft 3020 about a rotation axis O and relative to fifth shaft 3026/fourth gear 3028. Rotation axis O may be substantially perpendicular to rotation axis M and may be substantially parallel to a central longitudinal axis of reusable portion 3002.

In use, rotation of second control knob 3024 about rotation axis M in a direction (e.g., clockwise or counterclockwise) may cause third shaft 3014/second gear 3018 to also rotate about rotation axis M in the same direction. Rotation of third shaft 3014/second gear 3018 about rotation axis M may cause second gear 3018 to mesh with fourth gear 3030, and thereby rotate fourth gear 3030/fifth shaft 3026 about rotation axis O and relative to fourth shaft 3020/third gear 3028.

Disposable portion 3006 may include a first shaft 3032 and a second shaft 3034 within housing 3008. In this example, first shaft 3032 may be disposed with second shaft 3034. For example, first and second shafts 3032, 3034 may be concentric. First shaft 3032 may extend from a proximal end 3035 to a distal end that is fixedly coupled (e.g., rotationally fixedly coupled) to a first gear 3036 (e.g., a bevel gear). Second shaft 3034 may extend from a proximal end 3037 to a distal end that is fixedly coupled (e.g., rotationally fixedly coupled) to a second gear 3038 (e.g., a bevel gear). First gear 3036 and second gear 3038 may be coaxial with first gear 3036 being distal to second gear 3038. First shaft 3032/first gear 3036 may rotate independently of second shaft 3034/second gear 3038.

As shown in FIG. 12, proximal end 3035 of first shaft 3032 may be distal to proximal end 3037 of second shaft 3034, such that second shaft 3034 may define a lumen 3050. First shaft 3032 may define a lumen 3048 in communication with lumen 3050. A cross-sectional shape of lumen 3050 may be complementary to a cross-sectional shape of fifth shaft 3026, such that fifth shaft 3026 may be keyed into lumen 3050. Consequently, rotation of fourth gear 3030/fifth shaft 3026 about rotation axis O in a direction (e.g., clockwise or counterclockwise) may cause rotation of second shaft 3034/second gear 3038 about rotation axis O in the same direction. A cross-sectional shape of lumen 3048 may be complementary to a cross-sectional shape of distal portion 3023 of fourth shaft 3020, such that distal portion 3023 of fourth shaft 3020 may be keyed into lumen 3048. Consequently, rotation of third gear 3028/fourth shaft 3020 about rotation axis O in a direction (e.g., clockwise or counterclockwise) may cause rotation of first shaft 3032/first gear 3036 about rotation axis O in the same direction. In other aspects, other keying mechanisms may be used (e.g., circumferential teeth around each shaft).

Second shaft 3034 may define a proximal opening in communication with lumen 3050, which may help facilitate easy engagement or disengagement of fourth and fifth shafts 3020, 3026 with or from first and second shafts 3032, 3034, respectively, when coupling or decoupling reusable portion 3002 to or from disposable portion 3006. When reusable portion 3002 is coupled to disposable portion 3006, distal portion 3023 of fourth shaft 3020 may be received within lumen 3048 of first shaft 3032, and at least a distal portion (including distal end 3029) of fifth shaft 3026 may be received within lumen 3050 of second shaft 3034. Distal end 3029 of fifth shaft 3026 may abut proximal end 3035 of first shaft 3032.

First shaft 3032/first gear 3036 may be coupled to a third gear 3040 (e.g., a bevel gear) within housing 3008. Third gear 3040 may be positioned in contact with and substantially perpendicular to first gear 3036. Third gear 3040 may be fixedly coupled (e.g., rotationally fixedly coupled) to a first spool 3017. Disposable portion 3006 may include a first set of two pull wires 3044 coupled to and extending distally from first spool 3017, through the shaft of disposable portion 3006, and to the distal tip of the disposable portion 3006. Rotation of third gear 3040 about rotation axis S in a direction (e.g., clockwise or counterclockwise) may also rotate first spool 3017 about rotation axis S in the same direction, which in turn may move pull wires 3044 (e.g., move wires 3044 proximally or distally). Rotation axis S may be substantially perpendicular to rotation axis O and may be substantially parallel to rotation axis M.

Second shaft 3034/second gear 3038 may be coupled to a fourth gear 3042 (e.g., a bevel gear) within housing 3008. Fourth gear 3042 may be positioned in contact with and substantially perpendicular to second gear 3038. Fourth gear 3042 may be fixedly coupled (e.g., rotationally fixedly coupled) to a second spool 3019. Disposable portion 3006 may include a second set of two pull wires 3046 coupled to and extending distally from second spool 3019, through the shaft of disposable portion 3006, and to the distal tip of the disposable portion 3006. Rotation of fourth gear 3042 about rotation axis S in a direction (e.g., clockwise or counterclockwise) may also rotate second spool 3019 about rotation axis S in the same direction, which in turn may move pull wires 3046 (e.g., move wires 3046 proximally or distally).

As shown in FIG. 12, third gear 3040 and fourth gear 3042 may be disposed within and on opposite sides of housing 3008. First spool 3017 may be disposed on a side of third gear 3040 furthest from a center of housing 3008. In other examples, first spool 3017 may be disposed on a side of third gear 3040 closest to the center of housing 3008. Similarly, as shown in FIG. 12, second spool 3019 may be disposed on a side of fourth gear 3042 furthest from the center of housing 3008. In other examples, second spool 3019 may be disposed on a side of fourth gear 3042 closest to the center of housing 3008. Although third gear 3040/first spool 3017 and fourth gear 3042/second spool 3019 are described as having the same rotation axis S, in other examples, third gear 3040/first spool 3017 and fourth gear 3042/second spool 3019 may not be coaxial.

In use, an operator may rotate first control knob 3022 about rotation axis M in a direction (e.g., clockwise or counterclockwise), which may rotate second shaft 3012/first gear 3016 about rotation axis M in the same direction. Rotation of second shaft 3012/first gear 3016 about rotation axis M may cause third gear 3028/fourth shaft 3020 to rotate about rotation axis O in a direction (e.g., clockwise or counterclockwise) and relative to fourth gear 3030/fifth shaft 3026, which in turn may cause first shaft 3032/first gear 3036 to also rotate about rotation axis O in the same direction and relative to second shaft 3034/second gear 3038. Rotation of first shaft 3032/first gear 3036 about rotation axis O may cause third gear 3040/first spool 3017 (e.g., a spool assembly) to rotate about rotation axis S, which in turn may move pull wires 3044 (e.g., proximally or distally) and thereby deflect the distal tip of disposable portion 3006 in the left or right direction.

In use, an operator may rotate second control knob 3024 about rotation axis M in a direction (e.g., clockwise or counterclockwise), which may rotate third shaft 3014/second gear 3018 about rotation axis M in the same direction. Rotation of third shaft 3014/second gear 3018 about rotation axis M may cause fourth gear 3030/fifth shaft 3026 to rotate about rotation axis O in a direction (e.g., clockwise or counterclockwise) and relative to third gear 3028/fourth shaft 3020, which in turn may cause second shaft 3034/second gear 3038 to also rotate about rotation axis O in the same direction and relative to first shaft 3032/first gear 3036. Rotation of second shaft 3034/second gear 3038 about rotation axis O may cause fourth gear 3042/second spool 3019 (e.g., a spool assembly) to rotate about rotation axis S, which in turn may move pull wires 3046 (e.g., move pull wires 3046 proximally or distally) and thereby deflect the distal tip of disposable portion 3006 in the up or down direction.

In alternatives, first control knob 3022 may control up/down movement, and second control knob 3024 may control left/right movement. Rotation of first and second control knobs 3022, 3024 in an opposite direction to that described above may result in opposite directions of rotation of the other elements described above.

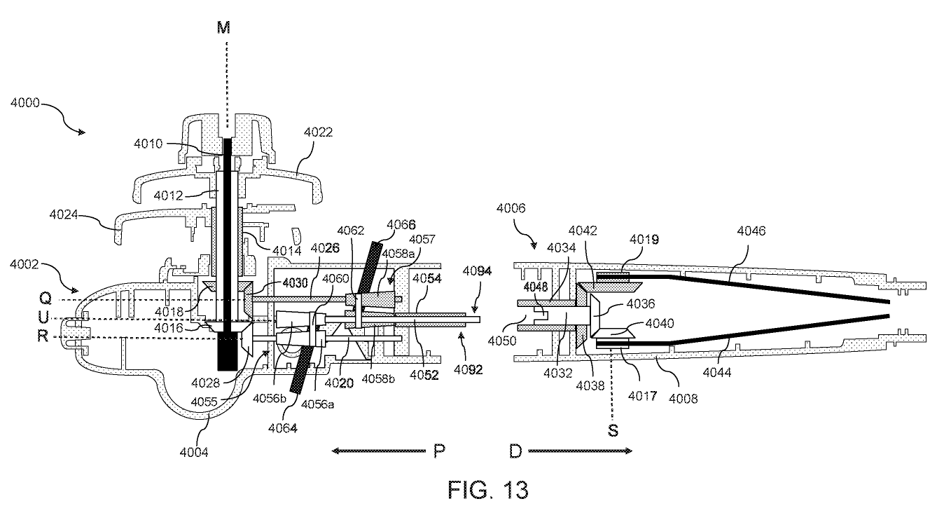

FIG. 13 illustrates a schematic, partially cross-sectional view of features of an exemplary medical device 4000 that includes a reusable portion 4002 and a disposable portion 4006. Reusable portion 4002 may include a housing 4004 configured to be removably coupled to a housing 4008 of disposable portion 4006 to form a handle of medical device 4000. Reusable portion 4002 may include any of the features of reusable portions 201, 301, 401, 501, 901, 1002, 2002, 3002, unless otherwise specified herein, and disposable portion 4006 may include any of the features of disposable portions 203, 903, 1006, 2006, 3006, unless otherwise specified herein. For example, disposable portion 4006 may include a shaft having any of the features of shaft 206. The shaft of disposable portion 4006 may extend distally from housing 4008 to a deflectable distal tip (similar to distal tip 208).

Reusable portion 4002 may include a first control knob 4022 similar to first control knobs 1022, 2022, 3022 for deflecting the distal tip of disposable portion 4006 in a left or right direction, and may include a second control knob 4024 similar to second control knobs 1024, 2024, 3024 for deflecting the distal tip of disposable portion 4006 in an up or down direction. Reusable portion 4002 may include a first shaft 4010 (e.g., a mounting shaft) similar to first shafts 1010, 2010, 3010, a second shaft 4012 (similar to second shafts 1012, 2012, 3012) coupled to first control knob 4022, and a third shaft 4014 (similar to third shafts 1014, 2014, 3014) coupled to second control knob 4024. Second shaft 4012 may be fixedly coupled (e.g., rotationally fixedly coupled) to a first gear 4016 (e.g., a bevel gear) similar to first gears 1016, 2016, and third shaft 4014 may be fixedly coupled (e.g., rotationally fixedly coupled) to a second gear 4018 (e.g., a bevel gear) similar to second gears 1018, 2018. For example, first gear 4016 may surround first shaft 4010, and second gear 4018 may surround second shaft 4012. First and second gears 4016, 4018 may be coaxial.

Reusable portion 4002 may further include a fourth shaft 4020, a fifth shaft 4026, a sixth shaft 4052, and a seventh shaft 4054 within housing 4004. Each of shafts 4020, 4026, 4052, 4054 may extend substantially parallel to a longitudinal axis of housing 4004. Fourth shaft 4020 may be fixedly coupled (e.g., rotationally fixedly coupled) to a third gear 4028 (e.g., a bevel gear). For example, a proximal end of fourth shaft 4020 may be fixedly coupled to third gear 4028. Fifth shaft 4026 may be fixedly coupled (e.g., rotationally fixedly coupled) to a fourth gear 4030 (e.g., a bevel gear). For example, a proximal end of fifth shaft 4026 may be fixedly coupled to fourth gear 4030.

Third gear 4028/fourth shaft 4020 may be gearingly coupled to first gear 4016/second shaft 4012. Third gear 4028 may be positioned in contact with and substantially perpendicular to first gear 4016, such that first gear 4016 may mesh with third gear 4028 to transfer torque to fourth shaft 4020 at an approximately 90-degree angle. Fourth gear 4030/fifth shaft 4026 may be gearingly coupled to second gear 4018/third shaft 4014. Fourth gear 4030 may be positioned in contact with and substantially perpendicular to second gear 4018, such that second gear 4018 may mesh with fourth gear 4030 to transfer torque to fifth shaft 4026 at an approximately 90-degree angle.

Fourth shaft 4020 may be rotatably coupled to sixth shaft 4052 via a first belt assembly 4055. First belt assembly 4055 may include a first cone or frustum 4056a, a second cone or frustum 4056b, a belt 4060, and an actuator 4064. First frustum 4056a may be fixedly coupled (e.g., rotationally fixedly coupled) to fourth shaft 4020. Second frustum 4056b may be fixedly coupled (e.g., rotationally fixedly coupled) to sixth shaft 4052. For example, second frustum 4056b may be fixedly coupled to a proximal end of sixth shaft 4052, and positioned adjacent to first frustum 4056a. First and second frustums 4056a, 4056b may taper in opposite directions. For example, first frustum 4056a may taper to a smaller diameter/width in a proximal direction, and second frustum 4056b may taper to a smaller diameter/width in a distal direction. Belt 4060 may be wrapped around first and second frustums 4056a, 4056b. Belt 4060 may be movable, such that rotation of first frustum 4056a causes movement of belt 4060 and, as a result, movement of second frustum 4056b. Belt 4060 may also be axially/longitudinally movable in a direction parallel to a central longitudinal axis of reusable portion 4002 (e.g., movable in a proximal or distal direction) along and relative to first and second frustums 4056a, 4056b. Actuator 4064 (e.g., a lever, a motor) may be coupled to belt 4060 and may be configured to move belt 4060 proximally or distally along first and second frustums 4056a, 4056b.

Fifth shaft 4026 may be rotatably coupled to seventh shaft 4054 via a second belt assembly 4057. Second belt assembly 4057 may include a first cone or frustum 4058a, a second cone or frustum 4058b, a belt 4062, and an actuator 4066. First frustum 4058a may be fixedly coupled (e.g., rotationally fixedly coupled) to fifth shaft 4026. Second frustum 4058b may be fixedly coupled (e.g., rotationally fixedly coupled) to seventh shaft 4054. For example, second frustrum 4058b may be fixedly coupled to a proximal end of seventh shaft 4054, and positioned adjacent to first frustrum 4058a. First and second frustums 4058a, 4058b may taper in opposite directions. For example, first frustum 4058a may taper to a smaller diameter/width in a proximal direction, and second frustum 4058b may taper to a smaller diameter/width in a distal direction. Belt 4062 may be wrapped around first and second frustums 4058a, 4058b. Belt 4062 may be movable, such that rotation of first frustum 4058a causes movement of belt 4062 and, as a result, movement of second frustum 4058b. Belt 4060 may also be axially/longitudinally movable in a direction parallel to the central longitudinal axis of reusable portion 4002 (e.g., movable in a proximal or distal direction) along and relative to first and second frustums 4058a, 4058b. Actuator 4066 (e.g., a lever, a motor) may be coupled to belt 4062 and may be configured to move belt 4062 proximally or distally along first and second frustums 4058a, 4058b. As will be discussed in greater detail below, belts 4060, 4062 may be longitudinally movable along frustums 4056a, 4056b and frustums 4058a, 4058b, respectively, to change gear ratios during a medical procedure. In some aspects, actuators 4064, 4066 or belts 4060, 4062 may be maintained in a desired position through use of friction, a ratcheting catch mechanism, or other similar mechanism as would be understood by those skilled in the art.

As shown in FIG. 13, first belt assembly 4055 may be disposed proximally to second belt assembly 4057 to accommodate first and second belt assemblies 4055, 4057 in a constrained space of housing 4004 and the arrangement of sixth shaft 4052 and seventh shaft 4054, described below. Sixth shaft 4052 may extend distally from second frustum 4056b, through second frustum 4058b, through seventh shaft 4054, and to a distal portion 4094. Distal portion 4094 of sixth shaft 4052 may be distal to a distal end 4092 of seventh shaft 4054. Sixth shaft 4052 (and second frustum 4056b) and seventh shaft 4054 (and second frustum 4058b) may be coaxial. For example, sixth and seventh shafts 4052, 4054 may rotate about a rotation axis U independently of one another. A cross-sectional shape of seventh shaft 4054 may be any suitable non-round/non-circular cross-sectional shape, and a cross-sectional shape of distal portion 4094 of sixth shaft 4052 may have any non-round/non-circular cross-sectional shape. In some examples, sixth and seventh shafts 4052, 4054 may be spline shafts. In alternatives, sixth and seventh shafts 4052, 4054 may be parallel but not coaxial, similar to the arrangements of medical devices 1000 and 2000.

Disposable portion 4006 may include a first shaft 4032, a second shaft 4034, a first gear 4036, a second gear 4038, a third gear 4040, a fourth gear 4042, a first spool 4017, a second spool 4019, a first set of two pull wires 4044, and a second set of two pull wires 4046 similar to first shaft 3032, second shaft 3034, first gear 3036, second gear 3038, third gear 3040, fourth gear 3042, first spool 3017, second spool 3019, first set of two pull wires 3044, and second set of two pull wires 3046, respectively. For example, first shaft 4032, second shaft 4034, first gear 4036, second gear 4038, third gear 4040, fourth gear 4042, first spool 4017, second spool 4019, first set of two pull wires 4044, and second set of two pull wires 4046 may be arranged in a similar or same manner as described above for first shaft 3032, second shaft 3034, first gear 3036, second gear 3038, third gear 3040, fourth gear 3042, first spool 3017, second spool 3019, first set of two pull wires 3044, and second set of two pull wires 3046.

In this example, first shaft 4032 may define a lumen 4048 having a cross-sectional shape complementary to a cross-sectional shape of distal portion 4094 of sixth shaft 4052, and second shaft 4034 may define a lumen 4050 having a cross-sectional shape complementary to a cross-sectional shape of seventh shaft 4054. As discussed above, a cross-sectional shape of each of distal portion 4094 of sixth shaft 4052 and seventh shaft 4054 may be non-round/non-circular. Consequently, distal portion 4094 of sixth shaft 4052 may be keyed into lumen 4048 of first shaft 4032, and seventh shaft 4054 may be keyed into lumen 4050 of second shaft 4034 when reusable portion 4002 is coupled to disposable portion 4006.

In use, the operator may rotate first control knob 4022 about rotation axis M in a direction (e.g., clockwise or counterclockwise), which may rotate second shaft 4012/first gear 4016 about rotation axis M in the same direction. Rotation of second shaft 4012/first gear 4016 about rotation axis M may cause third gear 4028/fourth shaft 4020 to rotate about a rotation axis R in a direction (e.g., clockwise or counterclockwise), which in turn may cause first frustum 4056a to rotate, thereby causing belt 4060 to move and to rotate second frustum 4056b to rotate sixth shaft 4052 about rotation axis U in the same direction and relative to second frustrum 4058b/seventh shaft 4054. Due to distal portion 4094 of sixth shaft 4052 being keyed into lumen 4048 of first shaft 4032, first shaft 4032/first gear 4036 may also rotate about rotation axis U in the same direction (relative to second shaft 4034/second gear 4038). Rotation of first shaft 4032/first gear 4036 about rotation axis U may cause third gear 4040/first spool 4017 (e.g., a spool assembly) to rotate about rotation axis S to move pull wires 4044 (e.g., move pull wires 4044 proximally or distally) and thereby deflect the distal tip of disposable portion 4006 in the left or right direction.

In use, the operator may rotate second control knob 4024 about rotation axis M in a direction (e.g., clockwise or counterclockwise), which may rotate third shaft 4014/second gear 4018 about rotation axis M in the same direction. Rotation of third shaft 4014/second gear 4018 about rotation axis M may cause fourth gear 4030/fifth shaft 4026 to rotate about a rotation axis Q in a direction (e.g., clockwise or counterclockwise), which in turn may cause first frustum 4058a to rotate, thereby causing belt 4062 to move, and rotate second frustum 4058b to rotate seventh shaft 4054 about rotation axis U in the same direction and relative to second frustum 4056b/sixth shaft 4052. Due to seventh shaft 4054 being keyed into lumen 4050 of second shaft 4034, second shaft 4034/second gear 4038 may also rotate about rotation axis U in the same direction (relative to first shaft 4032/first gear 4036). Rotation of second shaft 4034/second gear 4038 about rotation axis U may cause fourth gear 4042/second spool 4019 (e.g., a spool assembly) to rotate about rotation axis S and move pull wires 4046 (e.g., move pull wires 4046 proximally or distally), and thereby deflect the distal tip of disposable portion 4006 in the up or down direction.

Rotation axes Q, U, and R may be substantially parallel to one another. Rotation axes Q, U, and R may each be substantially parallel to the central longitudinal axis of reusable portion 4002, and may each be substantially perpendicular to rotation axis M. Rotation axis S may be substantially perpendicular to rotation axes Q, U, and R, and may be substantially parallel to rotation axis M.

In alternatives, first control knob 4022 may control up/down movement, and second control knob 4024 may control left/right movement. Rotation of first and second control knobs 4022, 4024 in an opposite direction to that described above may result in opposite directions of rotation of the other elements described above.

As mentioned above, belt 4060 or belt 4062 may be moved longitudinally along frustums 4056a, 4056b or frustums 4058a, 4058b, respectively, to change a gear ratio associated with first control knob 4022 or second control knob 4024, respectively. The arrangement of frustums 4056a, 4056b and the arrangement of frustums 4058a, 4058b may provide an operator with a range of gear ratios (e.g., values ranging from high to low and all values in between) rather than discrete levels to select from (e.g., high, low, or neutral). The arrangements of frustums 4056a, 4056b and frustums 4058a, 4058b illustrated in FIG. 13 provide gear ratio ranges that increase in a distal direction. Alternatively, first frustums 4056a, 4058a may taper to become narrower in a distal direction, and second frustums 4056b, 4058b may taper to become narrower in a proximal direction to provide gear ratio ranges that decrease in a distal direction. If desired, the operator may gradually increase or decrease a gear ratio associated with first control knob 4022 or second control knob 4024 during a medical procedure. Belt 4060 is illustrated in a first position (e.g., a high gear ratio position) in which a diameter of first frustum 4056a is larger than a diameter of second frustum 4056b. Belt 4062 is illustrated in a second position (e.g., a low gear ratio position) in which a diameter of first frustum 4058a is smaller than a diameter of second frustum 4058b.

Due to first and second frustums 4056a, 4056b tapering in opposite directions, a diameter of first frustum 4056a and a diameter of second frustum 4056b may be different at any position of belt 4060, except for when belt 4060 is positioned at a midpoint (e.g., a neutral position, a 1:1 position) of first and second frustums 4056a, 4056b. Similarly, due to first and second frustums 4058a, 4058b tapering in opposite directions, a diameter of first frustum 4058a and a diameter of second frustum 4058b may be different at any position of belt 4062, except for when belt 4062 is at a midpoint (e.g., a neutral position, a 1:1 position) of first and second frustums 4058a, 4058b.

In use, due to a diameter of first frustum 4056a being larger than a diameter of second frustum 4056b in the configuration shown in FIG. 13, second frustum 4056b may rotate more than first frustum 4056a, which may result in greater articulation or deflection of the distal tip of disposable portion 4006 for a given rotation of knob 4022, but may require an operator to exert more force on first control knob 4022. An operator may change a position of belt 4060 from the position illustrated in FIG. 13 by actuating actuator 4064 to move belt 4060, e.g., proximally or distally relative to first and second frustums 4056a, 4056b, and thereby change a gear ratio between first and second frustums 4056a, 4056b.

In use, due to a diameter of first frustum 4058a being smaller than a diameter of second frustum 4058b in the configuration of FIG. 13, second frustum 4058b may rotate less than first frustum 4058b, which may result in less articulation or deflection of the distal tip of disposable portion 4006 for a given rotation of knob 4024, but may require an operator to exert less force on second control knob 4024. An operator may change a position of belt 4062 from the position illustrated in FIG. 13 by actuating actuator 4066 to move belt 4062, e.g., proximally or distally relative to first and second frustums 4058a, 4058b, and thereby change a gear ratio between first and second frustums 4058a, 4058b.

Alternatively, frustums 4056a, 4056b, 4058a, 4058b may each be replaced by a pulley lacking a frustum shape, and thus may not provide an operator with a range of gear ratios. In this alternative, actuators 4064 and 4066 may be omitted.

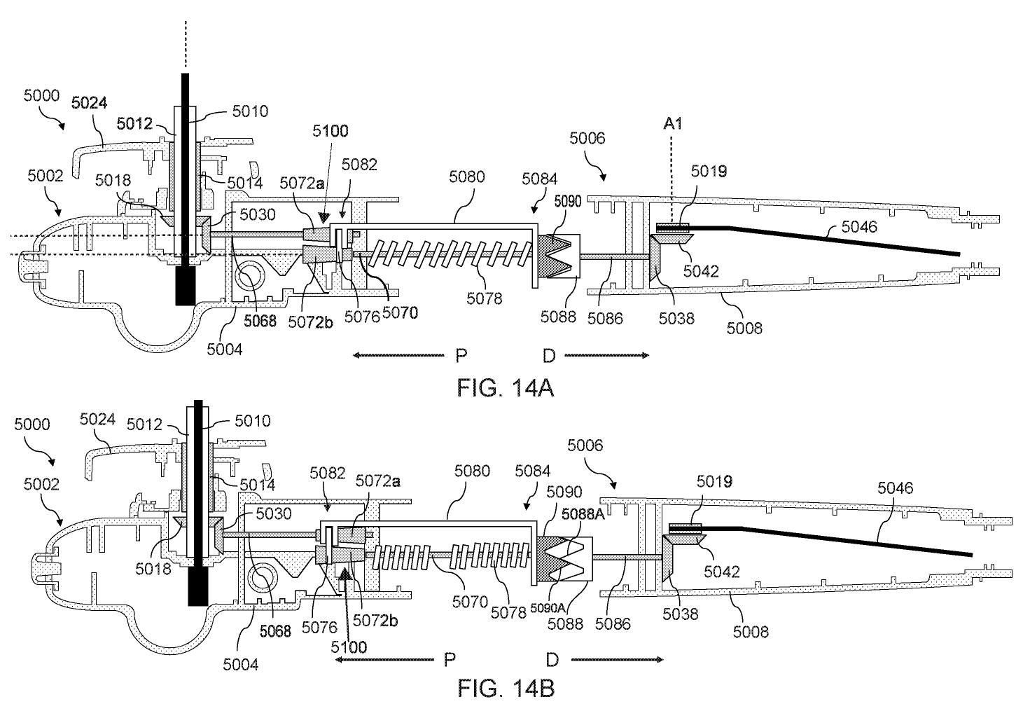

FIGS. 14A and 14B illustrate schematic, cross-sectional views of features of an exemplary medical device 5000 in a first state (FIG. 14A) and a second state (FIG. 14B). Medical device 5000 may include a reusable portion 5002 and a disposable portion 5006. Reusable portion 5002 may include a housing 5004 configured to be removably coupled to a housing 5008 of disposable portion 5006 to form a handle of medical device 5000. Reusable portion 5002 may include any of the features of reusable portions 201, 301, 401, 501, 901, 1002, 2002, 3002, 4002, unless otherwise specified herein, and disposable portion 5006 may include any of the features of disposable portions 203, 903, 1006, 2006, 3006, 4006, unless otherwise specified herein. For example, disposable portion 5006 may include a shaft having any of the features of shaft 206. The shaft of disposable portion 5006 may extend distally from housing 5008 to a deflectable distal tip (similar to distal tip 208).

Aspects of medical device 5000 are omitted in order to focus on the distinguishing features. Reusable portion 5002 may include a second control knob 5024 similar to second control knobs 1024, 2024, 3024, 4024 for deflecting the distal tip of disposable portion 5006 in an up or down direction. Reusable portion 5002 may also include a first control knob having any of the properties of the first control knobs described above (e.g., first control knobs 1022, 2022, 3022, 4022). The first control knob is not shown in order to focus on aspects of medical device 5000 that differ from other embodiments. Reusable portion 5002 may include a first shaft 5010 (e.g., a mounting shaft) similar to first shafts 1010, 2010, 3010, 4010, a second shaft 5012 similar to second shafts 1012, 2012, 3012, 4012 and coupled to the first control knob, and a third shaft 5014 similar to third shafts 1014, 2014, 3014, 4014 and coupled to second control knob 5024. Third shaft 5014 may be fixedly coupled (e.g., rotationally fixedly coupled) to a first gear 5018 (e.g., a bevel gear). For example, first gear 5018 may surround second shaft 5012.

Reusable portion 5002 may further include a fourth shaft 5068 and a fifth shaft 5070. Each of fourth and fifth shafts 5068, 5070 may extend substantially parallel to a longitudinal axis of housing 5004. Fourth shaft 5068 may be fixedly coupled (e.g., rotationally fixedly coupled) to a second gear 5030 and a first frustum 5072a. For example, a proximal end of fourth shaft 5068 may be fixedly coupled to second gear 5030, and a distal portion of fourth shaft 5068 may be fixedly coupled to first frustum 5072a. Second gear 5030/fourth shaft 5068/first frustum 5072a may be coupled to first gear 5018/third shaft 5014. Second gear 5030 may be positioned in contact with and substantially perpendicular to first gear 5018, such that first gear 5018 may mesh with second gear 5030 to transfer torque to fourth shaft 5068 at an approximately 90-degree angle.

Fourth shaft 5068 may be rotatably coupled to fifth shaft 5070 via a belt assembly 5100. Belt assembly 5100 may include first frustum 5072a, a second frustum 5072b, and a belt 5076. Second frustum 5072b may be fixedly coupled (e.g., rotationally fixedly coupled) to fifth shaft 5070. For example, second frustum 5072b may be fixedly coupled to a proximal end of fifth shaft 5070, and positioned adjacent to first frustum 5072a. First and second frustums 5072a, 5072b may taper in opposite directions. For example, first frustum 5072a may taper (narrow) in a proximal direction, and second frustum 5072b may taper (narrow) in a distal direction.

Belt 5076 may be wrapped around first and second frustums 5072a, 5072b. Belt 5076 may be movable, such that rotation of first frustum 5072a causes movement of belt 5076 and, as a result, movement of second frustum 5072b. Belt 5076 may also be axially/longitudinally movable in a direction parallel to a central longitudinal axis of reusable portion 5002 (e.g., movable in a proximal or distal direction) along and relative to first and second frustums 5072a, 5072b. Belt 5076 may be longitudinally movable along first and second frustums 5072a, 5072b to change a gear ratio between first and second frustums 5072a, 5072b. As discussed above for first and second belt assemblies 4055, 4057, the arrangement of first and second frustums 5072a, 5072b may provide an operator with a range of gear ratios (e.g., values ranging from high to low and all values in between). In this example, the arrangement of first and second frustums 5072a, 5072b provides a gear ratio range that increases in a distal direction. Alternatively, first frustum 5072a may taper (narrow) in a distal direction, and second frustum 5072b may taper (narrow) in a proximal direction to provide a gear ratio range that decreases in a distal direction.

Fifth shaft 5070 may extend distally from second frustum 5072b to a distal portion that is coupled to a coupler 5090. Fifth shaft 5070 may have a non-circular cross-sectional shape that is complementary to or keyed to a cross-sectional shape of an opening defined by coupler 5090. Alternatively, coupler 5090 may be coupled to fifth shaft 5070 by any suitable mechanism (e.g., retaining structures, etc.). Coupler 5090 may rotate with fifth shaft 5070/second frustum 5072b about a rotation axis W, but may also move longitudinally (e.g., in a proximal or distal direction) relative to fifth shaft 5070. Rotation axis W may be substantially parallel to the central longitudinal axis of reusable portion 5002, and may be substantially perpendicular to rotation axis M.

Reusable portion 5002 may further include a spring 5078 and a rod 5080 extending from a proximal end 5082 to a distal end 5084. Proximal end 5082 of rod 5080 may be fixedly coupled to belt 5076. Spring 5078 may surround fifth shaft 5070 and may be disposed proximally to coupler 5090. Distal end 5084 of rod 5080 may also surround fifth shaft 5070, such that distal end 5084 of rod 5080 may move axially or longitudinally relative to fifth shaft 5070.

Disposable portion 5006 may include a coupler 5088, a shaft 5086, a first gear 5038 (e.g., a bevel gear), a second gear 5042 (e.g., a bevel gear), and a set of two pull wires 5046 within housing 5008. Shaft 5086 may extend substantially parallel to a longitudinal axis of housing 5008. A proximal end of shaft 5086 may be fixedly coupled (e.g., rotationally fixedly coupled) to coupler 5088, and a distal end of shaft 5086 may be fixedly coupled (e.g., rotationally fixedly coupled) to first gear 5038 (e.g., a bevel gear).

Coupler 5090 of reusable portion 5002 may be removably engageable or connectable with coupler 5088 of disposable portion 5006. For example, a shape of coupler 5090 of reusable portion 5002 may be complementary to a shape of coupler 5088 of disposable portion 5006. Coupler 5090 may include a plurality of ramped surfaces 5090A, and coupler 5088 may include a plurality of ramped surfaces 5088A. Ramped surfaces 5090A and 5088B may be configured to contact one another. For example, couplers 5090 and 5088 may include teeth that mesh together.

As shown in FIGS. 14A and 14B, couplers 5090, 5088 may be fully engaged to one another (FIG. 14A) or partially engaged to one another (FIG. 14B). In either configuration, because the teeth or other mating structures of couplers 5090 and 5088 continue to mesh, rotation of coupler 5090 causes rotation of coupler 5088. Rotation of second frustum 5072b/fifth shaft 5070/coupler 5090 about rotation axis W in a direction (e.g., clockwise or counterclockwise) may cause rotation of coupler 5088/shaft 5086/first gear 5038 about rotation axis W in the same direction.

Coupler 5088/shaft 5086/first gear 5038 may be coupled to second gear 5042. Second gear 5042 may be fixedly coupled to (e.g., rotationally fixedly coupled) to spool 5019, and may be positioned in contact with and substantially perpendicular to first gear 5038. Pull wires 5046 may be coupled to and extend distally from spool 5019, through shaft 5011, and to the distal tip of disposable portion 5006. Rotation of coupler 5088/shaft 5086/first gear 5038 about rotation axis W may cause second gear 5042/spool 5019 to rotate about a rotation axis A1, which in turn may cause set of pull wires 5046 to move (e.g., move proximally or distally) and thereby deflect the distal tip of disposable portion 5006 in the up or down direction. Rotation axis A1 may be substantially perpendicular to rotation axis W, and may be substantially parallel to rotation axis M.

A gear ratio of belt assembly 5100 may automatically be adjusted in response to a tension on the shaft of disposable portion 5006. For example, in a highly tortuous body environment, in conventional scopes, control knobs become harder to rotate due to the bending of the shaft. Rotation of second frustrum 5072b, fifth shaft 5070, and coupler 5090 may exert a torsional force on coupler 5088 where ramped surfaces 5090A and 5088A contact one another. Torsional (circumferential) forces along ramped surfaces 5090A, 5088A may generate an axial force between couplers 5090 and 5088. As shown in FIG. 14B, the axial force may compress spring 5078 causing coupler 5090 and rod 5080, including proximal end 5082 of rod 5080, to move proximally. Proximal movement of proximal end 5082 of rod 5080 may cause belt 5076 to also move proximally along first and second frustums 5072a, 5072b. A decrease in the portion of belt 5076 on first frustum 5072a and an increase of the portion of the belt 5076 on second frustum 5072b may generate an increase in gear ratio and reduce the amount of torque in fourth shaft 5068 that matches the torque in fifth shaft 5070. Therefore, belt assembly 5100 may automatically adjust to the configuration shown in FIG. 14B, so that less force is required on knob 5024. In a more neutral state of the shaft of disposable portion 5006, belt assembly 5100 may automatically adjust to the configuration shown in FIG. 14A. An amount of force perceived by the operator may be the same or similar in the configurations of FIGS. 14A and 14B.

In use, an operator may rotate second control knob 5024 about rotation axis M in a direction (e.g., clockwise or counterclockwise), which may rotate third shaft 5014/first gear 5018 about rotation axis M in the same direction. Rotation of third shaft 5014/first gear 5018 about rotation axis M may cause second gear 5030/fourth shaft 5068 to rotate about a rotation axis L in a direction (e.g., clockwise or counterclockwise), which in turn may cause first frustum 5072a to rotate, thereby causing belt 5076 to move, and to rotate second frustrum 5072b to rotate fifth shaft 5070/coupler 5090 about rotation axis W in the same direction. Due to coupler 5090 being fully or partially engaged to coupler 5088, rotation of second frustum 5072b/fifth shaft 5070/coupler 5090 about rotation axis W may cause coupler 5088/shaft 5086/first gear 5083 to also rotate about rotation axis W in the same direction. Rotation of coupler 5088/shaft 5086/first gear 5083 about rotation axis W may cause second gear 5042/spool 5017 to rotate about rotation axis A1, which in turn may move pull wires 5046 (e.g., move pull wires 5046 proximally or distally) and thereby deflect the distal tip of disposable portion 5006 in the up or down direction. Rotation axis L may be substantially parallel to rotation axis W and the central longitudinal axis of reusable portion 5002, and may be substantially perpendicular to rotation axes M and A1.

As discussed above, during a procedure, belt assembly 5100 may automatically adjust to alter the gear ratio and to compensate for increased or decreased torque that would be required on knob 5024 in a device lacking belt assembly 5100.

Although not illustrated, the first control knob of reusable portion 5002 may be coupled to a similar mechanism as described above for second control knob 5024 to deflect the distal tip of shaft 5011 in a left or right direction. In alternatives, the first control knob may control up/down movement, and second control knob 5024 may control left/right movement. Rotation of the first control knob and second control knob 5024 in an opposite direction to that described above may result in opposite directions of rotation of the other elements described above.

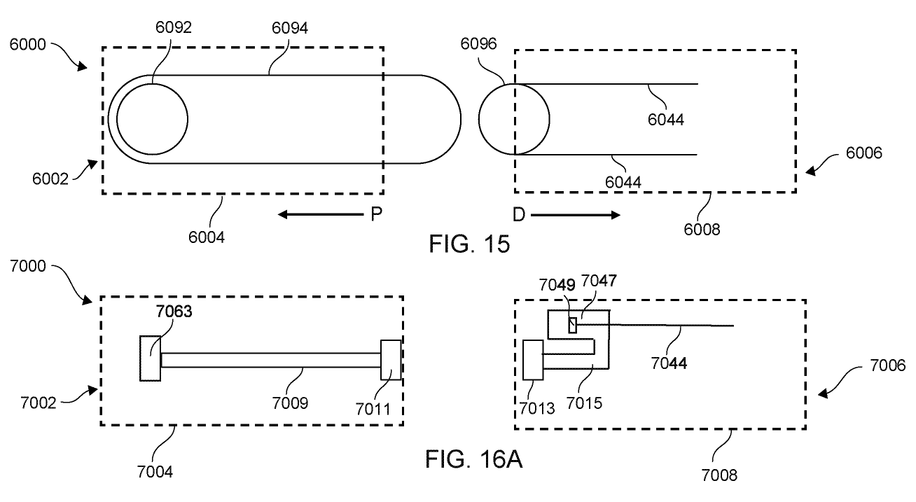

FIG. 15 illustrates a schematic view of features of an exemplary medical device 6000 including a reusable portion 6002 configured to be removably coupled to a disposable portion 6006. Reusable portion 6002 may include a housing 6004 configured to be removably coupled to a housing 6008 of disposable portion 6006 to form a handle of medical device 6000. Reusable portion 6002 may include any of the features of reusable portions 201, 301, 401, 501, 901, 1002, 2002, 3002, 4002, 5002, unless otherwise specified herein, and disposable portion 6006 may include any of the features of disposable portions 203, 903, 1006, 2006, 3006, 4006, 5006, unless otherwise specified herein. For example, disposable portion 6006 may include a shaft (similar to shaft 206) extending distally from housing 6008 to a deflectable distal tip (similar to distal tip 208).

In this example, reusable portion 6002 may include a pulley 6092 and a belt 6094 within housing 6004. Pulley 6092 may be fixedly coupled (e.g., rotationally fixedly coupled) to a first control knob (not shown) for deflecting the distal tip of disposable portion in a left or right direction. Belt 6094 may wrap around pulley 6092 and may extend distally past a distal end of housing 6004. Disposable portion 6006 may include a pulley 6096 coupled to a set of two pull wires 6044 that extend to the distal tip of disposable portion 6006. Pulley 6096 may be partially exposed from the proximal end of housing 6008. Belt 6094 may be partially exposed from the distal end of housing 6004, and may be configured to wrap around pulley 6096 to rotatably couple pulleys 6092, 6096. For example, belt 6094 may be removably coupled to pulley 6096 when reusable portion 6002 is coupled to disposable portion 6006.

In use, rotation of pulley 6092 about a rotation axis B1 in a direction (clockwise or counterclockwise) via the first control knob may cause belt 6094 to move around pulleys 6092, 6096 to rotate pulley 6096 about a rotation axis B2 in the same direction. Rotation of pulley 6096 about rotation axis B2 may move pull wires 6044 (e.g., move pull wires 6044 proximally or distally) and thereby deflect the distal tip of disposable portion 6006 in the left or right direction.

Although not illustrated, reusable portion 6002 may include a second control knob coupled to a similar mechanism as described above for the first control knob to deflect the distal tip of disposable portion 6006 in an up or down direction. For example, reusable portion 6002 may include a second pulley 6092 coupled to a second belt 6094 within housing 6004, and disposable portion 6006 may include a second pulley 6096 coupled to a second set of pull wires 6044 within housing 6008. The second belt 6094 may be removably coupled to the second pulley 6096.

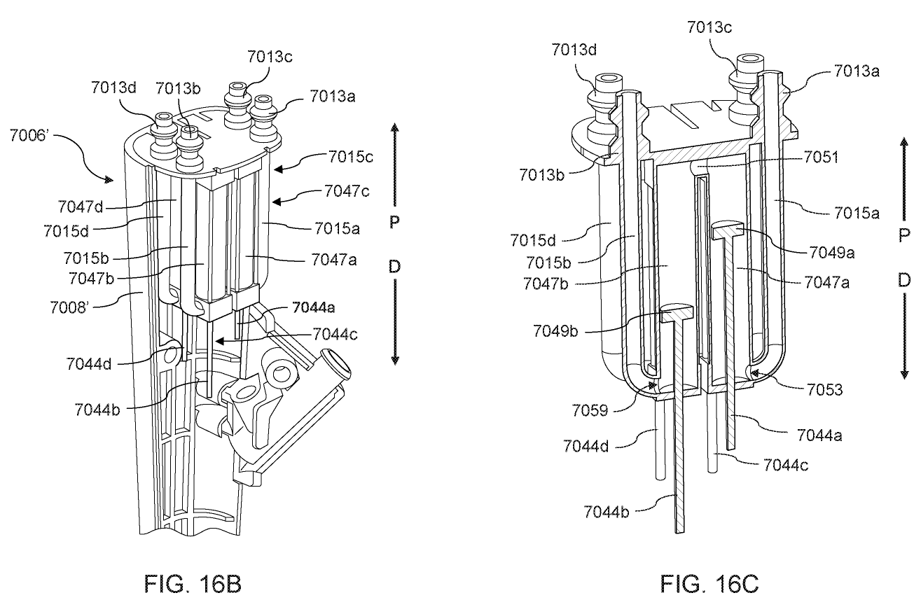

FIG. 16A illustrates a schematic view of features of an exemplary medical device 7000 including a reusable portion 7002 configured to be removably coupled to a disposable portion 7006. Reusable portion 7002 may include a housing 7004 configured to be removably coupled to a housing 7008 of disposable portion 7006 to form a handle of medical device 7000. Reusable portion 7002 may include any of the features of reusable portions 201, 301, 401, 501, 901, 1002, 2002, 3002, 4002, 5002, 6002, unless otherwise specified herein, and disposable portion 7006 may include any of the features of disposable portions 203, 903, 1006, 2006, 3006, 4006, 5006, 6006, unless otherwise specified herein. For example, disposable portion 7006 may include a shaft (similar to shaft 206) extending distally from housing 7008 to a deflectable distal tip (similar to distal tip 208). Reusable portion 7002 may include a first control knob (not shown but having any property of the knobs above) for left and right deflection of the distal tip of disposable portion 7006, and having any of the features of the first control knobs described above, unless otherwise specified herein. Reusable portion 7002 include a second control knob (not shown but having any property of the knobs above) for up and down deflection of the distal tip of disposable portion 7006, and having any of the features of the second control knobs described above, unless otherwise specified herein. While FIG. 16A illustrates one of each component 7009, 7011, 7013, 7015, 7044, 7047, 7049, and 7063, medical device 7000 may include four of each component 7009, 7011, 7013, 7015, 7044, 7047, 7049, and 7063, as will be discussed below.

In this example, medical device 7000 may include a hydraulic mechanism and/or a pneumatic mechanism for deflecting the distal tip of disposable portion 7006. Reusable portion 7002 may include four valves 7063 within housing 7004. Valves 7063 may be coupled to a pump for supplying fluid (e.g., air or water) to medical device 7000. Two of the four valves 7063 may be coupled to the first control knob, and the other two of the four valves 7063 may be coupled to the second control knob. Alternatively, the first control knob may be coupled to a first double-action valve, and the second control knob may be coupled to a second double-action valve.

Each valve 7063 may be coupled to a respective channel or conduit 7009 within housing 7004, and each channel 7009 may be coupled to a respective connector 7011 (e.g., a pneumatic or hydraulic plug, quick connector, or quick disconnector) at a distal end of housing 7004.

Disposable portion 7006 may include four connectors 7013 (e.g., a pneumatic or hydraulic socket, quick connector, or quick disconnector) complementary to a respective connector 7011 of reusable portion 7002. For example, each connector 7013 may be disposed at a proximal end of housing 7008. Each connector 7013 may be in fluid communication with a respective channel 7015 within housing 7008. The coupling between respective connectors 7011, 7013 may allow fluid to flow from a respective channel 7009 of reusable portion 7002 to a respective channel 7015 of disposable portion 7006. The coupling between connectors 7011 and 7013 may be, e.g., a quick release coupling that may facilitate easy connection or disconnection of channels 7009 and 7015, and thus easy connection or disconnection of reusable portion 7002 and disposable portion 7006.

Each channel 7015 may be in fluid communication with a respective chamber 7047 within housing 7008. Each chamber 7047 may include a piston 7049 that is fixedly coupled to a pull wire 7044 that extends to the distal tip of disposable portion 7006. Each piston 7049 may be movable within its respective chamber 7047, e.g., movable in a proximal or distal direction. Rotation of the first control knob or the second control knob or actuation of an alternative actuator may control delivery of fluid to its respective chambers 7047 to move its respective pistons 7049 and pull wires 7044 proximally or distally to deflect the distal tip of the disposable portion 7006.

FIGS. 16B and16C illustrate features of an exemplary disposable portion 7006’ that may be used in combination with reusable portion 7002, e.g., in place of disposable portion 7006, and may include any of the features of disposable portion 7006. For example, disposable portion 7006’ may include a shaft (similar to shaft 206) extending distally from a housing 7008’ to a deflectable distal tip (similar to distal tip 208). FIG. 16B illustrates disposable portion 7006’ with a portion of housing 7008’ removed to better illustrate internal components of housing 7008’. FIG. 16C illustrates a cross-sectional view of features of disposable portion 7006’. Disposable portion 7006’ may include four connectors 7013a, 7013b, 7013c, and 7013d (similar to connectors 7013), four channels 7015a, 7015b, 7015c, and 7015d (similar to channels 7015), four chambers 7047a, 7047b, 7047c, and 7047d (similar to chambers 7047), and four pull wires 7044a, 7044b, 7044c, and 7044d (similar to pull wires 7044). Each connector 7013a, 7013b, 7013c, 7013d may be configured to connect with a respective connector 7011 of reusable portion 7002.

Each pull wire 7044a, 7044b, 7044c, 7044d may be coupled to (e.g., fixedly coupled to) a respective piston (similar to piston 7049). For example, as shown in FIG. 16C, pull wire 7044a may be coupled to a piston 7049a that is disposed within chamber 7047a, and pull wire 7044b may be coupled to a piston 7049b that is disposed within chamber 7047b. Pistons 7049a, 7049b may be movable (e.g., movable proximally or distally) within chambers 7047a, 7047b, respectively. Pull wire 7044a may move with piston 7049a, and pull wire 7044b may move with piston 7049b. Although not shown, pull wire 7044c may be coupled to and movable with a piston within chamber 7047c, and pull wire 7044d may be coupled to and movable with a piston within chamber 7047d.

Connectors 7013a, 7013b, 7013c, 7013d may be in fluid communication with chambers 7047a, 7047b, 7047c, 7047d, respectively. Connector 7013a may in fluid communication with chamber 7047a via channel 7015a, connector 7013b may be in fluid communication with chamber 7047b via channel 7015b, connector 7013c may be in fluid communication with chamber 7047c via channel 7015c, and connector 7013d may be in fluid communication with chamber 7047d via channel 7015d. Channels 7015a, 7015b, 7015c, 7015d may each extend distally from its respective connector 7013a, 7013b, 7013c, 7013d to a distal end that curves inward toward a distal end of its respective chamber 7047a, 7047b, 7047c, 7047d.

As better shown in FIG. 16C, channel 7015a may be in fluid communication with chamber 7047a via a distal opening 7053 of chamber 7047a, and channel 7015b may be in fluid communication with chamber 7047b via a distal opening 7059 of chamber 7047b. Chamber 7047a may be in fluid communication with chamber 7047b via an opening 7051 between proximal ends of chambers 7047a and 7047b. Consequently, fluid may travel from connector 7013a to connector 7013b (and vice versa) to move pistons 7049a and 7049b in opposite directions.

Channels 7015c, 7015d and chambers 7047c, 7047d may be arranged in a similar manner described above for channels 7015a, 7015b and chambers 7047a, 7047b. Accordingly, chamber 7047c may be in fluid communication with chamber 7047d. Fluid may travel from connector 7013c to connector 7013d (and vice versa) to move the piston within chamber 7047c and the piston within chamber 7047d in opposite directions.

When reusable portion 7002 is coupled to disposable portion 7006’, connectors 7013a, 7013b, 7013c, 7013d of disposable portion 7006’ may each be coupled to a respective connector 7011 of reusable portion 7002. For example, connectors 7013a and 7013b may be coupled to respective connectors 7011 that are associated with the first control knob of reusable portion 7002, and connectors 7013c and 7013d may be coupled to respective connectors 7011 associated with the second control knob of reusable portion 7002.

In use and with reference to FIG. 16C, an operator may rotate the first control knob or actuate another actuator in a direction that may open one of its two respective valves 7063. Fluid provided by a pump may flow distally through a respective channel 7009 and respective connector 7011 of reusable portion, and through connector 7013a of disposable portion 7006’. The fluid may continue to flow distally through channel 7015a and through opening 7053. Once in chamber 7047a, the fluid may flow proximally towards a proximal end of chamber 7047a. As the fluid flows proximally, the fluid may exert an upward/proximal force on the bottom of piston 7049a causing piston 7049a and pull wire 7044a to move proximally. As a result, pull wire 7044a may be in a proximal (e.g., tensioned) state. Fluid may then flow through opening 7051 and distally through chamber 7047b. As the fluid flows distally, the fluid may exert a downward/distal force on piston 7049b causing piston 7049b and pull wire 7044b to move distally. As a result, pull wire 7044b may be in a distal (e.g., relaxed or loosened) state causing the distal tip of disposable portion 7006’ deflect in a left or right direction.

Excess fluid may then flow out of chamber 7047b, through opening 7059, and proximally through channel 7015b and connector 7013b. The excess fluid may continue to flow proximally through a respective connector 7013 and respective channel 7009 of reusable portion 7002, and back to the pump. Alternatively, fluid may be vented out via a vent hole. Rotation of the first control knob in an opposite direction may open the other valve of its respective two valves 7063, and may provide fluid that flows from connector 7013b to connector 7013a (e.g., fluid that flows in opposite directions described above) to deflect the distal tip of disposable portion 7006’ in a left or right direction.

The second control knob coupled to connectors 7013c and 7013d may control its respective valves 7063 in a similar manner described above for the first control knob. For example, fluid may flow from connector 7013c to 7013d (and vice versa) in a similar manner described above for connectors 7013a, 7013b to move the pistons within chambers 7047c and 7047d proximally or distally, and thus move pull wires 7044c, 7044d proximally or distally to deflect the distal tip of disposable portion 7006’ in an up or down direction. In an alternative, the first control knob may be coupled to connectors 7013c and 7013d, and the second control knob may be coupled to connectors 7013a and 7013b.

It will be appreciated that reusable portions 201, 301, 401, 501, 601, 901, 1002, 2002, 3002, 4002, 5002, 6002, 7002 may vary in shape, material, size or texture to, e.g., improve user comfort, gripping capabilities, or handling capabilities. For example, exterior surfaces of housings 205, 905, 1004, 2004, 3004, 4004, 5004, 6004, 7004 or exterior surfaces of gripping portions 783a-g may have different textures (e.g., smooth, rough, bumpy). Additionally, the reusable portions 201, 301, 401, 501, 601, 901, 1002, 2002, 3002, 4002, 5002, 6002, 7002 may vary in color. Reusable portions 201, 301, 401, 501, 601, 901, 1002, 2002, 3002, 4002, 5002, 6002, 7002 may be customized to a user in various ways, while maintaining compatibility with the disclosed disposable portions. The disclosed knobs may also be made of different materials (e.g., materials having different flexibilities or grip levels). The knobs may also have different shapes (e.g., asymmetrical shapes, shapes with ridges that protrude more or less, etc.).

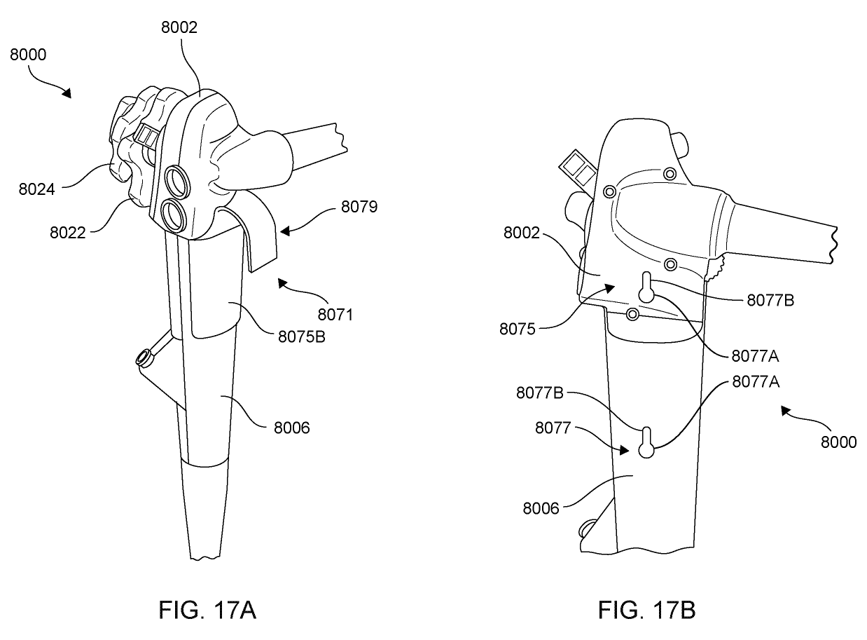

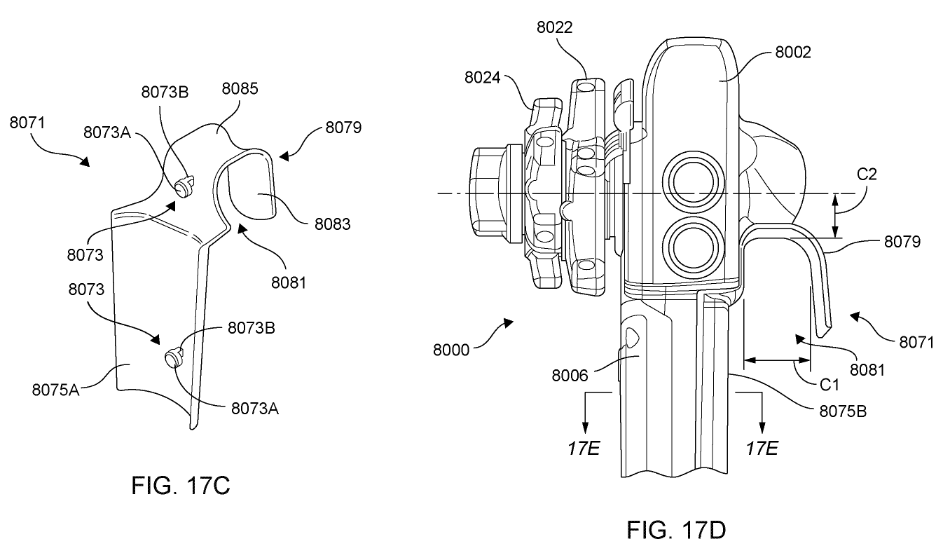

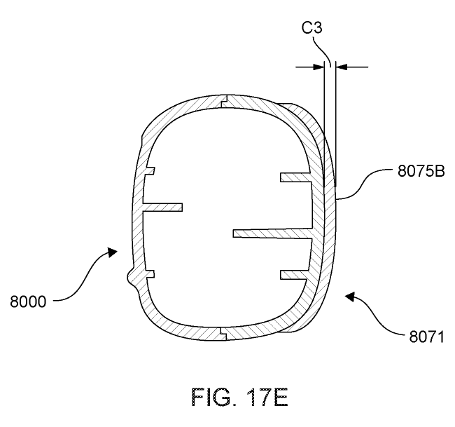

FIGS. 17A-17E illustrate features of an exemplary medical device 8000. Medical device 8000 may have any of the characteristics of any of the medical devices previously discussed. For example, medical device 8000 may include a reusable portion 8002 configured to be removably coupled to a disposable portion 8006. Together, reusable portion 8002 and disposable portion 8006 may form a handle of medical device 8000. Reusable portion 8002 may include any of the features of reusable portions 201, 301, 401, 501, 901, 1002, 2002, 3002, 4002, 5002, 6002, 7002, unless otherwise specified herein, and disposable portion 7006 may include any of the features of disposable portions 203, 903, 1006, 2006, 3006, 4006, 5006, 6006, 7006, unless otherwise specified herein.

In some aspects, medical device 8000 may include features that may be customizable according to the user. In aspects, such features may be configured for attachment to the handle of medical device 8000, and be reusable or disposable. In other aspects, described herein, such features may be integrally formed with medical device 8000.

For example, medical device 8000 may include a grip 8071. Grip 8071 may be secured to one or both of reusable portion 8002 or disposable portion 8006. Grip 8071 may be fixed to the handle of medical device 8000 (for example, on an opposite side of first control knob 122 and second control knob 124 of FIG. 1A).

Grip 8071 may be removably secured to medical device 8000 via one or more protrusions 8073 extending laterally outward from a first surface 8075A of grip 8071. First surface 8075A may be sized or shaped to abut against an outer surface of the handle of medical device 8000 without significant gaps between the two components. Each protrusion 8073 may include a first portion 8073A having a first diameter, and a second portion 8073B having a second, larger diameter. Each protrusion 8073 may be received within a respective locking recess 8075 defined on an outer surface of reusable portion 8002 or disposable portion 8006.

Each locking recess 8077 may include a first recess portion 8077A joined to a second recess portion 8077B. First recess portion 8077A may be larger in diameter as compared to second recess portion 8077B. In such a way, first portion 8073A of each protrusion 8073 may be received by first recess portion 8077A, and second recess portion 8077B may receive second portion 807B of each protrusion 8073.

To secure grip 8071 to medical device 8000, first portion 8073A of each protrusion may be inserted into a first recess portion 8077A of a respective locking recess 8075. Grip 8071 may then slide proximally relative to medical device 8000 such that second portion 8073B of each protrusion 8073 is received within second recess portion 8077B of a respective locking recess 8075.

Grip 8071 may be removably secured to medical device 8000 in other ways. For example, grip 8071 may be secured to medical device 8000 using fasteners (e.g., screws, bolts, etc.), a snap-fit, a press-fit, a strap that surrounds a diameter medical device, etc. In other aspects, grip 8071 may be permanently secured to medical device 8000 using adhesives, permanent fasteners, or other means to secure two components together permanently.

In further aspects, grip 8071 may be integrally formed with medical device 8000. For example, grip 8071 may be molded with the handle shells defining reusable portion 8002 or disposable portion 8006.

Grip 8071 may include a hook 8079 that extends laterally from a second surface 8075B of grip 8071. For example, second surface 8075B may be opposite first surface 8075A of grip 8071. Accordingly, hook 8079 may define a space 8081 to receive a user’s hand. For example, hook 8079 may be sized and shaped to rest between a user’s pointer finger and thumb. In aspects, hook 8079 may include a convex surface 8083 opposite a concave surface 8085. Such surfaces may facilitate a secure and comfortable grip between the user’s pointer finger and thumb.

All or portions of grip 8071 may be formed from a rigid material. In such aspects, grip 8071 may be provided in multiple sizes. The sizes of each grip 8071 may be determined by a width C1 of space 8081, a distance C2 between an axis of rotation of one or more control knobs 8022, 8024, and a thickness C3 of grip 8071, or any combination thereof.

In other aspects, grip 8071 may be formed of a rigid material based on a user’s specific anatomy or desired measurements for the width C1 of space 8081, the distance C2 between the axis of rotation of control knobs 8022, or the thickness C3 of grip 8071. The thickness C3 of grip 8071 may increase an overall diameter of medical device 8000, thus providing a wider grip. In some aspects, user-specific images or measurements may be provided such that grip 8071 is manufactured to the exact size or shape of the intended user in a rigid material.

In other aspects, all or portions of grip 8071 may be formed from a soft, pliable material capable of being bent or molded to fit a user’s hand. In such aspects, grip 8071 may be molded or bent according to the user’s needs for each procedure. For example, space 8081 may be increased or decreased by bending hook 8079 away from or towards second surface 8075B. In these aspects, grip 8071 may be customized in the field or between different users.

In other aspects, grip 8071 may include any of the features of gripping portions 783a-g described above. For example, exterior surfaces of grip 8071 may have different textures (e.g., smooth, rough, bumpy). For example, grip 8071 may partially or completely cover a circumference of the handle of medical device 800. For example, grip 8071 may vary in thickness.

It will be apparent to those skilled in the art at various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

What is claimed is:

1. A medical device comprising:
a first portion comprising:
a housing;
an actuator;
a gear assembly coupled to the actuator; and
a second portion removably coupled to the first portion, the second portion comprising:
a housing connected to the housing of the first portion;
a shaft extending distally from the housing of the second portion and defining a working channel; and
a spool assembly coupled to a first pull wire and a second pull wire,
wherein each of the first pull wire and the second pull wire extend through the shaft to a distal end of the shaft, and
wherein the actuator is coupled to the spool assembly via the gear assembly to deflect the distal end of the shaft.

2. The medical device of claim 1, wherein each of the first portion and the second portion include an umbilicus, wherein the umbilicus of the first portion is configured to transmit electrical signals, receive electrical signals, or deliver power to the medical device, and wherein the umbilicus of the second portion is configured to couple to a source of fluid or a source of suction.

3. The medical device of claim 1, wherein the spool assembly includes a spool and a gear, and wherein the gear of the spool assembly is fixedly coupled to the spool.

4. The medical device of claim 3, wherein the gear assembly comprises a first gear, a second gear, and a third gear, wherein the first gear is fixedly coupled to the actuator, wherein the second gear is disposed between and engaged with the first gear and the third gear, and wherein the third gear is disposed between and engaged with the second gear and the gear of the spool assembly.

5. The medical device of claim 4, wherein the first gear is aligned with the second gear and the third gear.

6. The medical device of claim 4, wherein the first gear is offset from the second gear and the third gear.

7. The medical device of claim 3, wherein the gear assembly comprises a first gear and a gear unit comprising a plurality of gears, wherein the first gear is fixedly coupled to the actuator, and wherein the gear unit is disposed between the first gear and the gear of the spool assembly.

8. The medical device of claim 7, wherein the gear unit comprises a first compound gear, a second compound gear, and a single gear disposed between the first compound gear and the second compound gear.

9. The medical device of claim 7, wherein the gear unit is laterally movable relative to the first gear to adjust a gear ratio of the gear assembly.

10. The medical device of claim 3, wherein the gear assembly comprises a first gear and a rack gear, wherein the first gear is fixedly coupled to the actuator, wherein a proximal end of the rack gear is engaged with the first gear, and wherein a distal end of the rack gear is engaged with the gear of the spool assembly.

11. The medical device of claim 1, wherein the first portion further comprises a first shaft within the housing of the first portion, wherein the gear assembly comprises a first gear and a second gear positioned perpendicularly to one another, wherein the first gear is fixedly coupled to the actuator, and wherein the second gear is fixedly coupled to the first shaft.

12. The medical device of claim 11, wherein the second portion further comprises a second shaft within the housing of the second portion and coupled to the spool assembly, and wherein the second shaft is rotatable with the first shaft to rotate the spool assembly.

13. The medical device of claim 12, wherein a cross-sectional shape of the first shaft is non-round, wherein the second shaft defines a lumen having a cross-sectional shape complementary to the cross-sectional shape of the second shaft, and wherein the first shaft is received within the lumen of the second shaft.

14. The medical device of claim 12, wherein the first portion further comprises a first coupler fixedly coupled to a distal end of the first shaft, wherein the second portion further comprises a second coupler fixedly coupled to a proximal end of the second shaft, and wherein the first coupler is engaged with the second coupler.

15. The medical device of claim 11, wherein the first portion further comprises a second shaft and a belt assembly within the housing of the first portion, wherein the second portion further comprises a third shaft coupled to the spool assembly within the housing of the second portion, wherein the first shaft is coupled to the second shaft via the belt assembly, and wherein the third shaft is rotatable with the second shaft to rotate the spool assembly.

16. A medical device comprising:
a first portion comprising:
an actuator; and
a first gear rotatably fixedly coupled to the actuator; and
a second portion removably coupled to the first portion, the second portion comprising:
a shaft;
a spool assembly comprising a second gear fixedly coupled to a spool;
a first pull wire; and
a second pull wire,
wherein each of the first pull wire and the second pull wire extend distally from the spool to a distal end of the shaft, and
wherein the actuator is coupled to the spool assembly to deflect the distal end of the shaft.

17. The medical device of claim 16, wherein the first portion further comprises a third gear and a fourth gear, wherein the third gear is disposed between the first gear and the fourth gear, and wherein the fourth gear is disposed between the third gear and the second gear.

18. The medical device of claim 16, wherein the first portion further comprises a rack gear, and wherein the rack gear is engaged with the first gear and the second gear.

19. The medical device of claim 16, wherein the first portion further comprises a third gear and a first shaft extending parallel to a longitudinal axis of the first portion, wherein the third gear is positioned in contact with and perpendicular to the first gear, and wherein the second portion further comprises a second shaft coupled to the first shaft and the spool assembly.

20. A medical device comprising:
a first portion comprising:
a housing;
an actuator;
a first gear within the housing of the first portion and rotatably fixedly coupled to the actuator; and
a second gear that engages with the first gear; and
a second portion removably coupled to the first portion, the second portion comprising:
a third gear within the housing of the second portion; and
a pull wire;
wherein the pull wire is coupled to the third gear; and
wherein the first gear is rotatable via the actuator to indirectly rotate the third gear to actuate the pull wire.
